(19) United States
(12) Patent Application Publication
(10) Pub. No.: US 2012/0178081 A1
(43) Pub. Date: Jul. 12, 2012

(54) Methods of Labeling Cells, Labeled Cells, and uses Thereof

(75) Inventors: Quan N. Nguyen, San Ramon, CA (US); Robert J. Lipshutz, Palo Alto, CA (US); Yunqing Ma, San Jose, CA (US)

(73) Assignee: Affymetrix. Inc., Santa Clara, CA (US)

(21) Appl. No.: 13/342,912

(22) Filed: Jan. 3, 2012

Related U.S. Application Data
(60) Provisional application No. 61/429,045, filed on Dec. 31, 2010.

(51) Int. Cl. G01N 33/53

(52) U.S. Cl. 435/6.11

(57) ABSTRACT

Methods of detecting nucleic acids, proteins and cells including methods of detecting two or more nucleic acids, proteins and cells in multiplex bDNA assays, are provided. Assays may be conducted at least in vitro, in vivo, in cellulo, and in situ. Nucleic acids are detected, through cooperative hybridization that results in specific association of a label probe system with target nucleic acids. Embodiments are directed to concurrent detection of one or more nucleic acids and/or one or more proteins. The detected proteins may be intracellular or external markers on the surface of the cell. Detection of protein components is accomplished by use of specific antibodies and a label probe system and/or coated microparticles which bind to the outside surface of specific cells and contain specific probes that can be detected using the same label probe system. Compositions, kits, and systems related to the methods are also described.

### PRIORITY CLAIM

This U.S. patent application claims priority from U.S. provisional application Ser. No. 61/429,045 filed on Dec. 31, 2010, the subject matter of which is incorporated herein by reference in its entirety.

### FIELD OF THE INVENTION

Disclosed are labeled cells, methods of labeling cells, compositions and kits for labeling and detection of cells, as well as simultaneous differentiation of one or more cell types. Detection may be, for instance, in vitro, in vivo, in cellulo and/or in situ. Detection may include or be directed towards detection of, for example, a nucleic acid, a protein, or combinations thereof. Any type of nucleic acid may be detected in the cell(s), such as siRNA, miRNA, mRNA, or DNA and analogues thereof. Cells may be labeled either internally or externally and then detected using a variety of signal detection methods. High-throughput analysis of large numbers of different cells may be achieved using the present methods and compositions, for instance by employing flow cytometry, microfluidic devices, filtration methods and fluorescence-activated cell sorting devices and like methods. Assays enable simultaneous detection of multiple different cells or cell types in a single sample in a robust and specific manner and optionally in a high-throughput mode.

### BACKGROUND OF THE INVENTION

The ability to differentially label, detect and quantitate cells has become very important in the medical fields for their value in diagnostics, as prognostic indicators of disease and determination of efficacy of disease treatments in patients. Human diseases such as cancer, infectious diseases, neurological disorders, muscular disorders and other diseases can be analyzed by examining the morphology and phenotype of cells. To aide in identifying and counting cells in human samples, several technologies such as Fluorescence Activated Cell Sorting (FACS) analysis have been developed and proven to be extremely helpful in the continuing battle against these diseases. To win the war against such dangerous diseases, there is a continuing need for advancement of tools and technologies which allow quicker, more efficient, more sensitive and accurate, and more effective means of analyzing cells. Cells may be analyzed in a number of ways, by either examining cell surface markers and proteins, or inner-workings of cells including genes, proteins and cellular structure/organization. Presented herein are technologies, methods and compositions, developed to differentially label cellular components to provide rapid, sensitive and accurate analyses of cells in disease and in health.

A variety of techniques for detection of nucleic acids involve a first step of capturing or binding of the target nucleic acid or nucleic acids to a surface through hybridization of each nucleic acid to an oligonucleotide (or other nucleic acid) that is attached to the surface. For example, DNA microarray technology, which is widely used to analyze gene expression, copy number determination and single nucleotide polymorphism detection, relies on hybridization of DNA targets to preformed arrays of polynucleotides. (See, e.g., Lockhart and Winzeler, “Genomics, gene expression and DNA arrays,” Nature, 405:827-36 (2000); Gerhold et al. “Monitoring expression of genes involved in drug metabolism and toxicology using DNA microarrays,” Physiol. Genomics, 5:161-70, (2001); Thomas et al. “Identification of toxicologically predictive gene sets using cDNA microarrays,” Mol. Pharmacol., 60:1189-94 (2001); and Epstein and Butow, “Microarray technology—enhanced versatility, persistent challenge,” Curr. Opin. Biotechnol., 11:36-41 (2000)). Single nucleotide polymorphism (SNP) has been used extensively for genetic analysis. Fast and reliable hybridization-based SNP assays have been developed. (See, Wang et al., Science, 280:1077-1082, 1998; Gingeras, et al., Genome Research, 8:435-448, 1998; and Halushka, et al., Nature Genetics, 22:239-247, 1999; incorporated herein by reference in their entireties). Methods and arrays for simultaneous genotyping of more than 10,000 SNPs, and more than 100,000 SNPs, have been described, for example, in Kennedy et al., Nat. Biotech., 21:1233-1237, 2003, Matsuzaki et al., Genome Res., 14(3):414-425, 2004, and Matsuzaki et al., Nature Methods, 1:109-111, 2004 (all of which are incorporated herein by reference in their entireties for all purposes).

Many different avenues of research have been investigated to address the issues of specificity and sensitivity of such hybridization-based genetic assays. For instance, the use of oligonucleotide analogs have been investigated which increase the melting temperature at which the target hybridizes to the capture oligonucleotide. New methods for hybridizing oligonucleotide probes in a specific manner with high affinity and desired sensitivity to target nucleic acids are constantly needed in the field of genetics research.

Global gene expression profiling and other technologies have identified a large number of genes whose expression is altered in diseased tissues or in tissues and cells treated with pharmaceutical agents. (See, Lockhart and Winzeler, (2000) “Genomics, gene expression and DNA arrays,” Nature, 405:827-36, and Gunther et al., (2003) “Prediction of clinical drug efficacy by classification of drug-induced genomic expression profiles in vitro,” Proc. Natl. Acad. Sci. USA, 100:9608-13). The capability of measuring the expression level of all of the expressed genes in a cell enables linking of these expression patterns to specific diseases. Therefore, gene expression is increasingly being used as a biomarker or prognosticator of disease, determination of the stage of disease, and indicator of prognosis. (See, Golub et al., (1999) “Molecular classification of cancer: class discovery and class prediction by gene expression monitoring,” Science, 286:531-7). Other applications of gene expression analysis and detection include, but are not limited to, target identification, validation and pathway analysis (Roberts et al. (2000) “Signaling and circuitry of multiple MAPK pathways revealed by a matrix of global gene expression profiles,” Science, 287:873-80), drug screening (Hamadeh et al., (2002) “Prediction of compound signature using high density gene expression profiling,” Toxicol. Sci., 67:232-40), and studies of drug efficacy, structure-activity relationship, toxicity, and drug-target interactions (Gerhold et al., (2001) “Monitoring expression of genes involved in drug metabolism and toxicology using DNA microarrays,” Physiol. Genomics, 5:161-70 and Thomas et al., (2001) “Identification of toxicologically predictive gene sets using cDNA microarrays,” Mol. Pharmacol., 60:1189-94). As biomarkers are identified, their involvement in disease management and drug development will need to be evaluated in higher throughput and broader populations of samples. Simpler and more flexible expression profiling technology that allows the expression analysis of multiple genes with higher data quality and higher throughput is therefore needed.

Often researchers desire information concerning both protein expression and transcription of DNA into messenger RNA. Though assays exist to separately detect mRNA and proteins, very few options exist for simultaneous detection of both species in a single sample. Further, no know methods exist for simultaneous detection of both mRNA and the encoded protein for multiple targets in a single sample. In situ assay of proteins to determine localization is traditionally achieved using immunochemical techniques. These traditional techniques use antibodies. When performing such assays as Fluorescence In Situ Hybridization (FISH), the tissue sample being analyzed is typically prepared in a very stringent manner, often destroying much of the protein information available in the cells. Thus, detection of proteins or enzymes using antibodies in concert with FISH techniques is incompatible and would yield mixed or inconsistent results at best. Other methods utilize traditional immunochemistry and isotope labeling. (See, Bursztajn et al., “Simultaneous visualization of neuronal protein and receptor mRNA,” Biotechniques, 9(4):440-449, 1990). Other techniques requiring much time-consuming manipulation and molecular genetic engineering utilize fluorescent proteins to perform the co-visualization. (See, Dahm et al., “Visualizing mRNA localization and local protein translation in neurons,” Methods Cell Biol., 85:293-327, 2008).

Simultaneous detection of both mRNA and translated protein allows comparison of the distribution of transcripts and corresponding expressed protein. This would allow visualization of where the protein products localize within the cell immediately following transcription. Furthermore, various mutants of the protein may be examined for changes in localization or half life depending on engineered transcript mutations, i.e. point mutations, truncations, fusions, and the like. Typically one would first perform immunohistochemical techniques to first visualize protein, followed immediately by attempted in situ hybridization to detect mRNA. However, the immunohistochemistry techniques often led to degradation of mRNA and weak mRNA signal in the second step. These steps may be reversed, but results are not consistent. One such method recently published uses DIG-based (dioxigenine-based) non-radioactive in situ hybridization on paraffin wax-embedded (FFPE) tissue sections, followed by immunohistochemistry. (See, Rex et al., “Simultaneous detection of RNA and protein in tissue sections by nonradioactive in situ hybridization followed by immunohistochemistry,” Biochemica, 3:24-26, 1994). However, FFPE is not suitable for every experimental investigation and often can perturb systems so that desired results are missed. It has long been recognized that FFPE samples can be difficult to work with and not desirable due to the extensive cross-linking which occurs during sample preparation and degradation and fragmentation of molecules caused by fixation. (See, Sahoo et al., J. Clin. Diag. Research, 3(3):1493-1499, 2009, citing Masuda et al., “Analysis of chemical modification of RNA from formalin fixed and optimizations of molecular biology applications for such samples,” Nucleic Acids Res., 27(22):4436-4443, 1999 and Quach et al., “In vitro mutation artifacts after formalin fixation and error prone translation synthesis during PCR,” BMC Clinical Pathology, 4:1, 2004). Thus, a need exists to find techniques that can reproducibly and quantitatively detect and localize both peptide and mRNA transcript species in a single sensitive assay in situ and in cellulo.

Levels of RNA expression have traditionally been measured using Northern blot and nuclease protection assays. However, these approaches are time-consuming and have limited sensitivity, and the data generated are more qualitative than quantitative in nature. Greater sensitivity and quantification are possible with reverse transcription polymerase chain reaction (RT-PCR) based methods, such as quantitative real-time RT-PCR, but these approaches have low multiplex capabilities. (See, Bustin, (2002) “Quantification of mRNA using real-time reverse transcription PCR (RT-PCR): trends and problems,” J. Mol. Endocrinol., 29:23-39, and Bustin and Nolan, (2004) “Pitfalls of quantitative real-time reverse-transcription polymerase chain reaction,” J. Biomol. Tech., 15:155-66). Microarray technology has been widely used in discovery research, but its moderate sensitivity and its relatively long experimental procedure have limited its use in high throughput expression profiling applications (Epstein and Butow, (2000) “Microarray technology—enhanced versatility, persistent challenge,” Curr. Opin. Biotechnol., 11:36-41).

Most of the current methods of mRNA quantification require RNA isolation, reverse transcription, and target amplification. Each of these steps has the potential of introducing variability in yield and quality that often leads to low overall assay precision. Recently, a multiplex screening assay for mRNA quantification combining nuclease protection with luminescent array detection was reported. (See, Martel et al., (2002) “Multiplexed screening assay for mRNA combining nuclease protection with luminescent array detection,” Assay Drug Dev. Technol., 1:61-71). Although this assay has the advantage of measuring mRNA transcripts directly from cell lysates, limited assay sensitivity and reproducibility were reported. Another multiplex mRNA assay without the need for RNA isolation was also reported in Tian et al., entitled “Multiplex mRNA assay using electrophoretic tags for high-throughput gene expression analysis.” (Nucleic Acids Res., 32:126, 2004). This assay couples the primary INVADER® mRNA assay with small fluorescent molecule Tags that can be distinguished by capillary electrophoresis through distinct charge-to-mass ratios of Tags. However, this assay requires the use of a specially designed and synthesized set of eTagged signal probes, complicated capillary electrophoresis equipment, and a special data analysis package.

Another genetic analysis product, called QUANTIGENE® (Panomics, Fremont, Calif.), is able to specifically bind and detect dozens of target sequences in a single sample. (See, for instance, U.S. patent application Ser. Nos. 11/433,081 (allowed), 11/431,092, 11/471,025 (allowed), all of which are incorporated herein by reference in their entirety for all purposes). General protocols and user's guides on how the QUANTIGENE® system works and explanation of kits and components may be found at the Panomics website (see, www.panomics.com/index.php?id=product_{—}1#product_lit_{—}1). Specifically, user's manual, “QUANTIGENE® 2.0 Reagent System User Manual,” (2007) provided at the Panomics website is incorporated herein by reference in its entirety for all purposes.

The QUANTIGENE® technology allows unparalleled signal amplification capabilities that provide an extremely sensitive assay. For instance, it is commonly claimed that the limit of detection in situ for mRNA species is about 20 copies of message per cell. However, in practice the limit of detection, due to the variability in the assay, is generally found to be around 50-60 copies of message per cell. This limit of detection limits the field of research since 80% of mRNAs are present at fewer than 5 copies per cell and 95% of mRNAs are present in cells at fewer than 50 copies per cell. As mentioned above, to arrive at this sensitivity, other approaches are very time consuming and complicated. Other technologies rely on the use of a panel of various enzymes and are affected by the fixation process of FFPE. In contrast, the QUANTIGENE® technology, such as QUANTIGENE® 2.0 and ViewRNA, is very simple, efficient and is capable of applying up to 400 labels per 50 base pairs of target. This breakthrough technology allows efficient and simple detection on the level of even a single mRNA copy per cell. Coupling this technology to detection of both mRNA and protein species will propel this field of research into heretofor inaccessible areas of study. The QUANTIGENE® assay has been proven to be robust, flexible, sensitive and accurate in many different types of applications, as exemplified and described in its many different forms, for instance, in U.S. Provisional Patent Application Ser. Nos. 61/360,887, 61/361,007 and 61/360,912 (all of which are incorporated by reference in their entirety for all purposes). There have additionally been allowed US patents encompassing a myriad number and types of branched-chain DNA detection-based assays, such as, for instance, U.S. Pat. Nos. 7,803,541, 7,709,198, 7,033,758, 6,232,462, 6,235,465, and 6,300,056 (all of which are incorporated by reference in their entirety for all purposes).

In addition to these various described methods of nucleic acid detection and quantitation, and protein detection and quantitation, and correlation thereof, there exists an extremely fruitful area of research dedicated to the detection and quantitation of specific cells or cell types. Of specific importance in the field of medical diagnostics is the detection and quantitation of specific tumor cells. Tumor cells may be circulating and detected, for instance, by circulating tumor cell (CTC) assays.

The process of cell sorting by FACS analysis is well established. The concept of flow cytometry provides the ability to count particles, such as cells, by passing them in solution through an electronic detection apparatus that is able to detect and quantitate various signals given by labels applied to the circulating cells or cells in suspension. (See, for instance, U.S. Pat. No. 2,656,508, incorporated herein by reference). Flow cytometry has also been referred to in the past as pulse cytophotometry, or cytofluorography. The principal of operation is fairly simple in that cells suspended in solution are passed through a flow cell. A beam of light, such as a laser beam, is directed at the flow cell as the cells pass by while one or more fluorescence detectors positioned about the flow cell capture signal events. The cells are labeled before being passed through the flow cell. Computers help to control the detectors and light sources and receive data and process the data accordingly. Modern flow cytometers comprise multiple wavelength lasers and fluorescence detectors and optionally provide for time of flight cell sorting capabilities, as in modern FACS machines. In FACS analysis, the stream of suspended cells is vibrated to produce droplets having a specific volume or dimension. Cells are then diluted such that there is a statistically great distance between cells allowing direction of the flow or spray into separate containers for collection. Alternative embodiments employ electrostatic deflection systems which divert droplets into containers based on their charge. Flow cytometry and labeling of cells allows for measurement of many important variables critical to diagnostics, therapeutics and other medically relevant exercises, such as, for instance, enzymatic activity, protein expression and localization, DNA copy number variation (flow-FISH applications), chromosome analysis, DNA and/or RNA content for instance for cell cycle analysis, cell viability, infection, apoptosis, transformation, various morphological variables, cell adherence, detection of the presence and/or absence of various cell surface markers indicative of disease, infection and cancer state, etc. A recent review of flow cytometry and its various applications is Perkel, J. M., “Ebb and Flow: Cytometry for the Next Generation,” Science, 330(6005):853-855, 2010 (incorporated herein by reference in its entirety for all purposes).

However, even today with all the available equipment and methods provided in advanced medical diagnostics, there continues to exist a dire need to expand these technology platforms to provide more robust and more sensitive indicators of disease and cell transformation. The assays that exist today are of limited scope and suffer from detection inefficiencies. The methodologies used are hampered by the availability of specific antibodies with the proper specificity, and the knowledge of a specific linkage between a specific cellular marker expressed on the surface of the cell and a specific disease or disease phenotype. None of the known methods allow simultaneous robust detection of many different markers including internal markers and including detection of both proteinaceous markers as well as nucleic acid markers.

Among other aspects, the present invention provides methods that overcome the above noted limitations and permit rapid, simple, and sensitive detection of multiple nucleic acids and proteins simultaneously in a population of cells for the purposes of quantitation and, for example, diagnosis of a disease state, prediction of disease progress, or as a prognosticator of therapeutic outcome. The presently disclosed compositions and methods allow for very sensitive and specific labeling of even heterogeneous cell populations by labeling either extracellular markers, intracellular markers, or both simultaneously, followed by rapid signal amplification and detection which may include high-throughput screening of the labeled cells.

### SUMMARY OF THE INVENTION

Disclosed are embodiments directed to labeling of a cell or multiple cells, wherein the cells may be identical or different. That is, the cell to be labeled may be a specific type of cell of interest present in a heterogeneous population of cells wherein the user wishes to label and detect the specific type of cell of interest based on differential labeling using a component which has specificity for an externally expressed antigen on the cell of interest. Multiple cells of interest may be labeled simultaneously using different types of labels allowing multiplexing of the assay. The methods, compositions, kits and systems of the present invention optionally utilize a microparticle barcode which has a visually detectable spatial code and optionally comprise the full bDNA assay set of components, or just certain parts of the labeling system as described in more detail below.

Presently provided are methods in which a sample which comprising or suspected of comprising a cell type of interest is incubated optionally with a microparticle, wherein the microparticle comprises a spatial code, a protein having affinity for an extracellular protein and a capture probe such that the protein binds the extracellular protein thereby associating the microparticle with the cell, and which is then incubated with one or more label extender probes and a label probe system such that the one or more label extender probes and label probe system hybridize with the capture probe, wherein the label extender probes comprise a sequence L-1 which is complementary to a sequence in the capture probe and a sequence L-2 complementary to a sequence found in a component of the label probe system, thereby labeling the cell.

Further provided are methods in which the sample comprises or is suspected of comprising at least two different cells and the label probe system comprises at least two different labels, each specific for each cell and/or wherein the label attached to the cells is detected by flow cytometry. The methods may be applied generally to almost any cell type, including but not limited to non-adherent and circulating cells. The method may also provide data such as the quantity of the label and/or the number of cells labeled and present in the sample.

The protein having affinity for an extracellular protein expressed or otherwise present on the surface of the cell of interest may generally be any protein having sufficient affinity to allow attachment or at least semi-permanent association of the microparticle with the cell. Thus, the protein having affinity for an extracellular protein may be an antibody and/or an antigen or mixtures and combinations thereof. The protein having affinity for an extracellular protein may be selected from one or more of the group consisting of an agonist, antagonist, phosphate-binding protein, saccharide-binding protein, and leptin-binding protein, for instance.

Furthermore, in the provided methods discussed above, the spatial code of the microparticle may be discernable by visual inspection and the cell or cells of interest may be generally any type of cell amenable to research or diagnostic application, such as, for instance, one or more of the group consisting of: stem cell, fibroblast, red blood cell, T cell, B cell, macrophage, lymphocyte, adipose cell, chondrocyte, and white blood cell and mixtures and combinations thereof.

Also presented are methods of labeling one or more cells or cell types of interest, wherein the methods include the steps of providing a sample comprising or suspected of comprising a cell, and incubating a protein having affinity for an extracellular protein with the sample such that the protein having affinity for an extracellular protein binds to the cell, wherein the protein having affinity for an extracellular protein comprises at least one amplifier or pre-amplifier probe sequence. In the present method, further including is the step of incubating at least one label probe system with the sample, thereby labeling the cell.

As in the previously discussed methods, in these methods the protein having affinity for an extracellular protein may generally be any protein known to have affinity for a component of the cell of interest present on the outer membrane of the cell of interest and therefore accessible by any other protein incubated therewith, such as, for instance, an antibody or an antigen.

Also provided are methods of labeling a cell which operate by providing a sample comprising or suspected of comprising a cell, incubating a microparticle comprising a spatial code with the sample, wherein the microparticle comprises a protein having affinity for an extracellular protein and one or more capture probes, such that the microparticle binds to the cell, and incubating the sample with one or more capture extenders, one or more target nucleic acids, one or more label extenders and one or more label probe systems such that the cell bound to the microparticle is labeled thereby labeling the cell.

In these methods, the sample may comprises or may be suspected of comprising at least two different cells and the label probe system therefore may comprises at least two different labels, each specific for each cell. Alternatively, the sample may comprise or may be suspected of comprising any number of cells or cell types of interest, such as 1, 2, 3, 4, 5, 6, 7, 8, 9, 10, 15, 20, 25, 30, 35, 40, 45, 50 or even 65 different cells or cell types of interest. Therefore the methods and assays may include as many, or more, different labels, and label probe system components as needed. As mentioned above, the label attached to the cells may be detected by flow cytometry and the cells may generally be any type of cell, such as but not limited to, non-adherent and circulating cells. Furthermore, the quantity of the label may be detected, thereby quantitating the number of cells labeled.

Additionally, with respect to these methods, as mentioned above, the protein having affinity for an extracellular protein may be an antibody and/or an antigen and mixtures and combinations thereof, such as one or more of the group consisting of: agonist, antagonist, phosphate-binding protein, saccharide-binding protein, and leptin-binding protein. The cell may be selected from one or more of the group consisting of: stem cell, fibroblast, red blood cell, T cell, B cell, macrophage, lymphocyte, adipose cell, chondrocyte, and white blood cell.

Also provided are methods of labeling a cell, wherein a sample comprising or suspected of comprising a cell or cell type of interest is incubated with an antibody specific for the target protein and wherein the antibody comprises at least one pre-amplifier probe sequence conjugated thereto. A label probe system may then be incubated with the sample and the cell detected and/or quantitated by detecting the presence or absence of the label. One or more components of the label probe system may optionally comprise one or more locked nucleic acids, such as but not limited to cEt. The assay enables localization and quantitation of the cell of interest, for instance within a tissue or within an organ. Label extenders may be designed in any number of different geometries, for instance as provided in FIG. 6.

Essentially all of the features noted for the embodiments above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

Further provided are labeling techniques including lanthanide isotopes and use of cytometric means with which to identify genes labeled in cells by time-of-flight inductively coupled plasma mass spectrometry (ICP-MS).

Further provided is evidence of bDNA assay employed on DNA microarrays.

### BRIEF DESCRIPTION OF THE DRAWINGS

FIG. 1 schematically illustrates a typical standard bDNA assay.

FIG. 2, Panels A-E schematically depict a multiplex nucleic acid detection assay, in which the nucleic acids of interest are captured on distinguishable subsets of microspheres and then detected.

FIG. 3A schematic of amplification multimer complex and labeling system for a cruciform structure label extender design. Note that this non-limiting depiction, as in others provided herein, only provides a single example of amplifier/pre-amplifier complex. In the assays, more or fewer amplifiers and label probes may be employed as needed.

FIG. 3B schematic of amplification multimer complex and labeling system for a “double z” or ZZ structure label extender design. Note that this non-limiting depiction, as in others provided herein, only provides a single example of amplifier/pre-amplifier complex. In the assays, more or fewer amplifiers and label probes may be employed as needed.

FIGS. 4A provides a depiction of a locked nucleic acid analog known as the constrained ethyl (cEt) nucleic acid analog. Note that as depicted various protecting groups known in the art are presented but may be substituted by any number of known suitable protecting groups.

FIGS. 4B depiction of a generic locked nucleic acid analog in the β-D, C3′-endo, conformation. The letter “B” stands for “base” which may be any one of A, G, C, mC, T or U. The methylene bridge connecting the 2′-O atom with the 4′-C atom is the chemical structure which “locks” the analog into the energy-favorable β-D conformation. However, it is understood that this bridge may be any number of carbon atoms in length and may contain any number of variable groups or substitutions as has been reported in the literature. Note also that, as depicted, various protecting groups known in the art are presented but may be substituted by any number of suitable protecting groups.

FIG. 5A schematically illustrates an encoded microparticle of the invention.

FIG. 5B is a side view cross-section of the microparticle in FIG. 5A.

FIG. 6 depicts various non-limiting conformations and geometries of label extender (LE) probes for detecting single stranded nucleic acid species. Other stereoisomers, conformers and various conformations are possible which achieve similar results but may not be depicted here. For convenience, the amplifiers and pre-amplifiers and label probes are not fully represented for all figures. The single line in light shading labeled as “label probe system” is meant to denote all possible configurations of label probe structures as described herein.

FIGS. 7A and 7B depict directionality of various label extenders and the possibility that label extenders may be designed in either direction, as indicated.

FIG. 8A illustrates the labeling of a cell with pre-amplifier conjugated to the substance which possesses specificity for an antigen found on the external surface of a cell.

FIG. 8B illustrates the labeling of a cell with a microparticle comprising a spatial barcode, a substance possessing affinity for a surface antigen on the cell (depicted as an antibody) and a capture probe, wherein the pre-amplifier associates with the capture probe through a label extender. This arrangement may optionally include a target nucleic acid captured to the capture probe by a capture extender, and the target nucleic acid hybridized to label extenders and the label probe system (not depicted here, see FIGS. 2-3).

FIG. 9 displays data obtained from performing bDNA assays on a DNA microarray. The x-axis provides the target gene identity and the y-axis represents relative light units. Samples were in vitro transcribed (IVT) in cell lysates. Two backgrounds were used (BK-a and Bk-b).

FIG. 10 provides cv analysis for three replicates of the genes of FIG. 9 all assayed on a single microarray.

FIG. 11 displays similar data as provided in FIG. 9 along with additional variable amounts of sample added as indicated in the legend.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a molecule” includes a plurality of such molecules, and the like.

The term “about” as used herein indicates the value of a given quantity varies by +/−10% of the value, or optionally +/−5% of the value, or in some embodiments, by +/−1% of the value so described.

The term “antibody” as referred to herein includes whole antibodies and any antigen binding fragment (i.e., “antigen-binding portion”) or single chains thereof. The term is meant to encompass all known isotypes of antibody, such as, for instance, IgG, IgA, IgD, IgE, and IgM. An “antibody” refers to a glycoprotein comprising at least two heavy (H) chains and two light (L) chains inter-connected by disulfide bonds, or an antigen binding portion thereof. The V_{H} and V_{L} regions of antibodies can be subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. That is, the term antibody is meant to encompass whole antibodies and fragments thereof that possess antigenic binding capability, such as, but not limited to, minibodies, diabodies, triabodies, tetrabodies, and the like. (See, for instance, Olafsen et al., Prot. Eng. Design and Selection, 17(4):315-323, 2004, Tramontano et al., J. Mol. Recognit., 7(1):9-24, 1994, and Todorovska et al., J. Immunol. Methods, 248(1-2):47-66, 2001). Furthermore, the term antibody is meant to encompass humanized antibodies or otherwise engineered antibodies which possess the desired antigen binding activity.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a F_{ab} fragment, a monovalent fragment consisting of the V_{L}, V_{H}, C_{L} and C_{H1} domains; (ii) a F(ab′)₂ fragment, a bivalent fragment comprising two F_{ab} fragments linked by a disulfide bridge at the hinge region; (iii) a F_{d} fragment consisting of the V_{H} and C_{H1} domains; (iv) a F′ᵥ fragment consisting of the V_{L} and V_{H} domains of a single arm of an antibody, (v) a dAb fragment (Ward et al., Nature, 341:544-546, 1989), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fᵥ (scFv); see e.g., Bird et al., Science, 242:423-426, 1988; and Huston et al., Proc. Natl. Acad. Sci. USA, 85:5879-5883, 1988). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “polynucleotide” (and the equivalent term “nucleic acid”) encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

The term “analog” in the context of nucleic acid analog is meant to denote any of a number of known nucleic acid analogs such as, but not limited to, LNA, PNA, etc. For instance, it has been reported that LNA, when incorporated into oligonucleotides, exhibit an increase in the duplex melting temperature of 2° C. to 8° C. per analog incorporated into a single strand of the duplex. The melting temperature effect of incorporated analogs may vary depending on the chemical structure of the analog, e.g. the structure of the atoms present in the bridge between the 2′-O atom and the 4′-C atom of the ribose ring of a nucleic acid.

For example, various bicyclic nucleic acid analogs have been prepared and reported. (See, for example, Singh et al., Chem. Commun., 1998, 4:455-456; Koshkin et al., Tetrahedron, 1998, 54:3607-3630; Wahlestedt et al., Proc. Natl. Acad. Sci. U.S.A., 2000, 97:5633-5638; Kumar et al., Bioorg. Med. Chem. Lett., 1998, 8:2219-2222; Wengel et al., PCT International Application Number PCT/DK98/00303 which published as WO 99/14226 on Mar. 25, 1999; Singh et al., J. Org. Chem., 1998, 63:10035-10039, the text of each is incorporated by reference herein, in their entirety). Examples of issued US patents and Published U.S. patent applications disclosing various bicyclic nucleic acids include, for example, U.S. Pat. Nos. 6,770,748, 6,268,490 and 6,794,499 and U.S. Patent Application Publication Nos. 20040219565, 20040014959, 20030207841, 20040192918, 20030224377, 20040143114, 20030087230 and 20030082807, the text of each of which is incorporated by reference herein, in their entirety.

Additionally, various 5′-modified nucleosides have also been reported. (See, for example: Mikhailov et al., Nucleosides and Nucleotides, 1991, 10:393-343; Saha et al., J. Org. Chem., 1995, 60:788-789; Beigleman et al., Nucleosides and Nucleotides, 1995, 14:901-905; Wang, et al., Bioorganic & Medicinal Chemistry Letters, 1999, 9:885-890; and PCT Internation Application Number WO94/22890 which was published Oct. 13, 1994, the text of each of which is incorporated by reference herein, in their entirety).

Oligonucleotides in solution as single stranded species rotate and move in space in various energy-minimized conformations. Upon binding and ultimately hybridizing to a complementary sequence, an oligonucleotide is known to undergo a conformational transition from the relatively random coil structure of the single stranded state to the ordered structure of the duplex state. With these physical-chemical dynamics in mind, a number of conformationally-restricted oligonucleotides analogs, including bicyclic and tricyclic nucleoside analogues, have been synthesized, incorporated into oligonucleotides and tested for their ability to hybridize. It has been found that various nucleic acid analogs, such as the common “Locked Nucleic Acid” or LNA, exhibit a very low energy-minimized state upon hybridizing to the complementary oligonucleotide, even when the complementary oligonucleotide is wholly comprised of the native or natural nucleic acids A, T, C, U and G.

Examples of issued US patents and published applications include for example: U.S. Pat. Nos. 7,053,207, 6,770,748, 6,268,490 and 6,794,499 and U.S. Patent Application Publication Nos. 20040219565, 20040014959, 20030207841, 20040192918, 20030224377, 20040143114 and 20030082807; the text of each of which is incorporated herein by reference, in their entirety for all purposes.

Additionally, bicyclo[3.3.0] nucleosides (bcDNA) with an additional C-3′,C-5′-ethano-bridge have been reported for all five of the native or natural nucleobases (G, A, T, C and U) whereas (C) has been synthesised only with T and A nucleobases. (See, Tarkoy et al., Helv. Chim. Acta, 1993, 76:481; Tarkoy and C. Leumann, Angew. Chem. Int. Ed. Engl., 1993, 32:1432; Egli et al., J. Am. Chem. Soc., 1993, 115:5855; Tarkoy et al., Helv. Chim. Acta, 1994, 77:716; M. Bolli and C. Leumann, Angew. Chem., Int. Ed. Engl., 1995, 34:694; Bolli et al., Helv. Chim. Acta, 1995, 78:2077; Litten et al., Bioorg. Med. Chem. Lett., 1995, 5:1231; J. C. Litten and C. Leumann, Helv. Chim. Acta, 1996, 79:1129; Bolli et al., Chem. Biol., 1996, 3:197; Bolli et al., Nucleic Acids Res., 1996, 24:4660). Oligonucleotides containing these analogues have been found to form Watson-Crick bonded duplexes with complementary DNA and RNA oligonucleotides. The thermostability of the resulting duplexes, however, is varied and not always improved over comparable native hybridized oligonucleotide sequences. All bcDNA oligomers exhibited an increase in sensitivity to the ionic strength of the hybridization media compared to natural counterparts.

A bicyclo[3.3.0] nucleoside dimer containing an additional C-2′,C-3′-dioxalane ring has been reported in the literature having an unmodified nucleoside where the additional ring is part of the internucleoside linkage replacing a natural phosphodiester linkage. As either thymine-thymine or thymine-5-methylcytosine blocks, a 15-mer polypyrimidine sequence containing seven dimeric blocks and having alternating phosphodiester- and riboacetal-linkages exhibited a substantially decreased Tₘ in hybridization with complementary ssRNA as compared to a control sequence with exclusively natural phosphordiester internucleoside linkages. (See, Jones et al., J. Am. Chem. Soc., 1993, 115:9816).

Other patents have disclosed various modifications of these analogs that exhibit the desired properties of being stably integrated into oligonucleotide sequences and increasing the melting temperature at which hybridization occurs, thus producing a very stable, energy-minimized duplex with oligonucleotides comprising even native nucleic acids. (See, for instance, U.S. Pat. Nos. 7,572,582, 7,399,845, 7,034,133, 6,794,499 and 6,670,461, all of which are incorporated herein by reference in their entirety for all purposes).

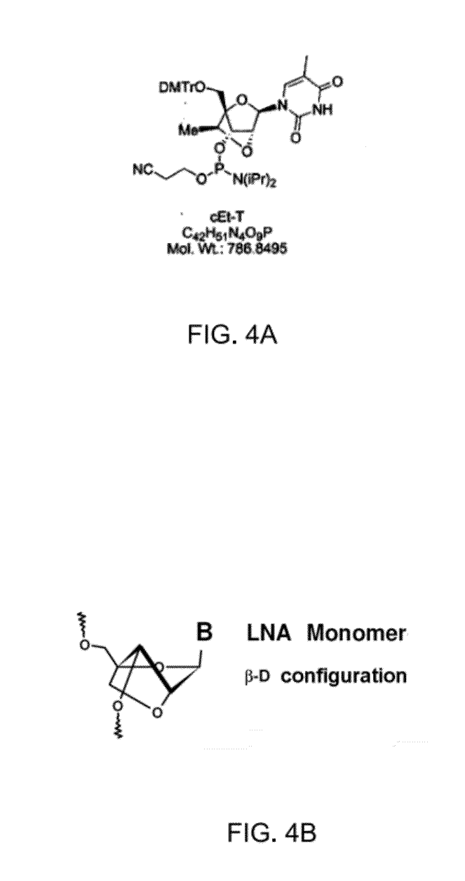

For instance, U.S. Pat. No. 7,399,845 provides 6-modified bicyclic nucleosides, oligomeric compounds and compositions prepared therefrom, including novel synthetic intermediates, and methods of preparing the nucleosides, oligomeric compounds, compositions, and novel synthetic intermediates. The '845 patent discloses nucleosides having a bridge between the 4′ and 2′-positions of the ribose portion having the formula: 2′-O—C(H)(Z)-4′ and oligomers and compositions prepared therefrom (see, for example, FIGS. 4A and 4B). In a preferred embodiment, Z is in a particular configuration providing either the (R) or (S) isomer, e.g. 2′-O,4′-methanoribonucleoside. It was shown that this nucleic acid analog exists as the strictly constrained N-conformer 2′-exo-3′-endo conformation. Oligonucleotides of 12 nucleic acids in length have been shown, when comprised completely or partially of the Imanishi et al. analogs, to have substantially increased melting temperatures, showing that the corresponding duplexes with complementary native oligonucleotides are very stable. (See, Imanishi et al., “Synthesis and property of novel conformationally constrained nucleoside and oligonucleotide analogs,” The Sixteenth International Congress of Heterocyclic Chemistry, Aug. 10-15, 1997, incorporated herein by reference in its entirety for all purposes).

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York), as well as in Ausubel, infra.

The “Tₘ” (melting temperature) of a nucleic acid duplex under specified conditions (e.g., relevant assay conditions) is the temperature at which half of the base pairs in a population of the duplex are disassociated and half are associated. The Tₘ for a particular duplex can be calculated and/or measured, e.g., by obtaining a thermal denaturation curve for the duplex (where the Tₘ is the temperature corresponding to the midpoint in the observed transition from double-stranded to single-stranded form).

The term “complementary” refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A “capture extender” or “CE” is a polynucleotide that is capable of hybridizing to a nucleic acid of interest and to a capture probe. The capture extender typically has a first polynucleotide sequence C-1, which is complementary to the capture probe, and a second polynucleotide sequence C-3, which is complementary to a polynucleotide sequence of the nucleic acid of interest. Sequences C-1 and C-3 are typically not complementary to each other. The capture extender is preferably single-stranded.

A “capture probe” or “CP” is a polynucleotide that is capable of hybridizing to at least one capture extender and that is tightly bound (e.g., covalently or noncovalently, directly or through a linker, e.g., streptavidin-biotin or the like) to a solid support, a spatially addressable solid support, a slide, a particle, a microsphere, or the like. The capture probe typically comprises at least one polynucleotide sequence C-2 that is complementary to polynucleotide sequence C-1 of at least one capture extender. The capture probe is preferably single-stranded.

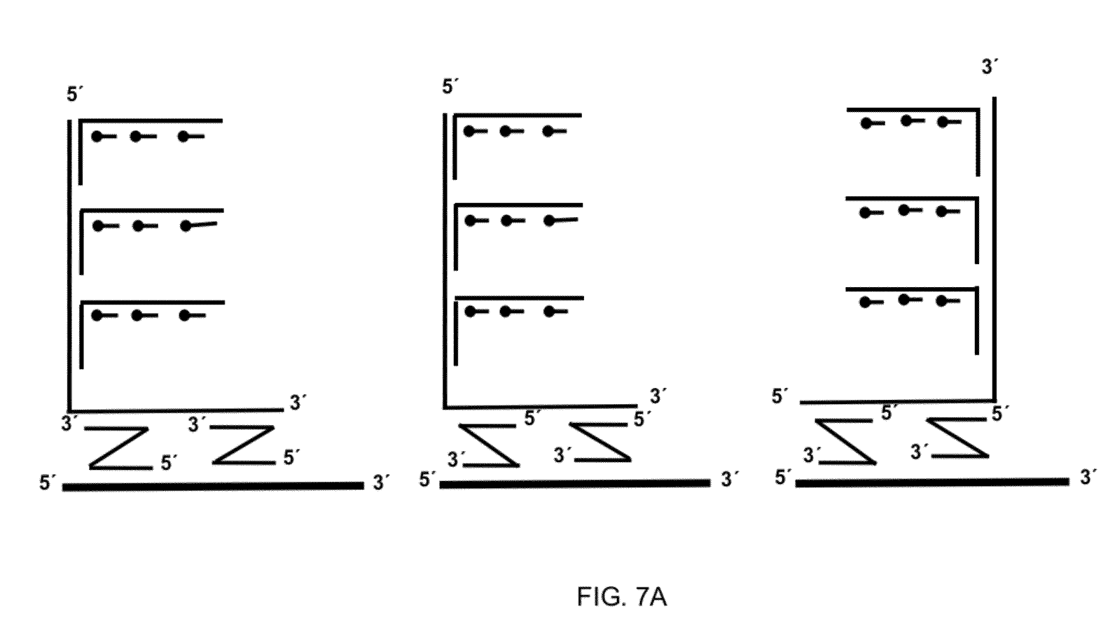

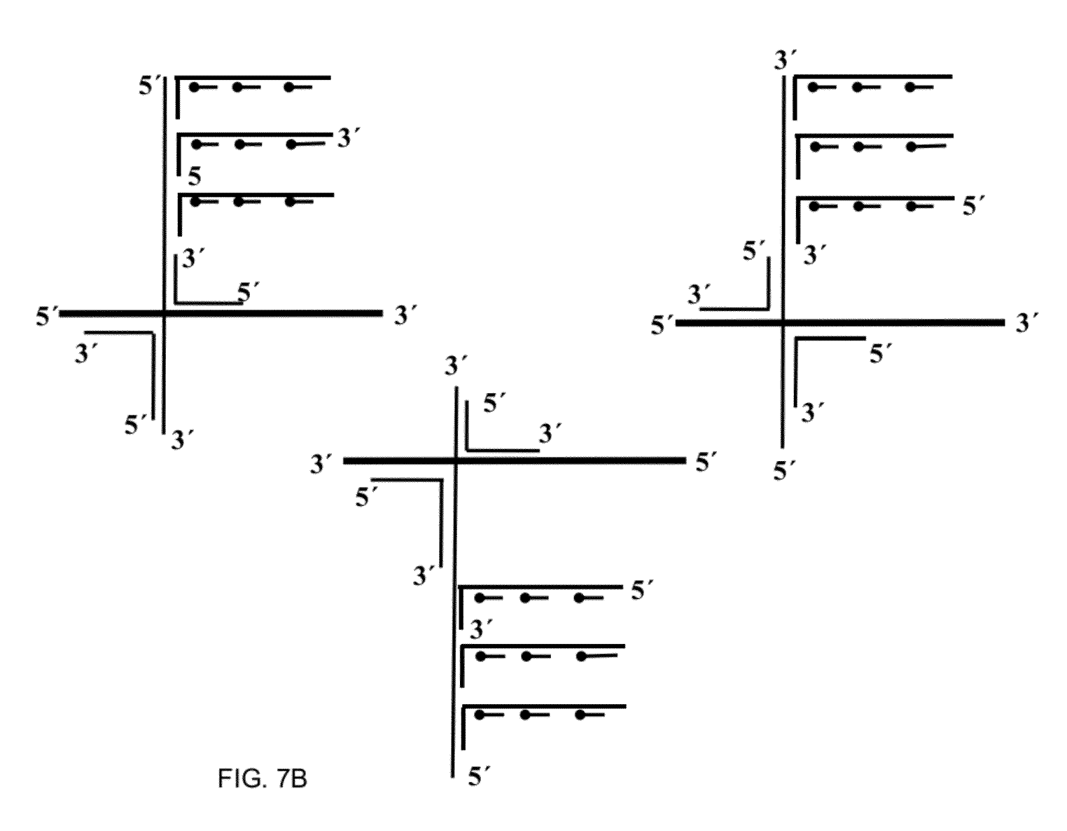

A “label extender” or “LE” is a polynucleotide that is capable of hybridizing to a nucleic acid of interest and to a label probe system. The label extender typically has a first polynucleotide sequence L-1, which is complementary to a polynucleotide sequence of the nucleic acid of interest, and a second polynucleotide sequence L-2, which is complementary to a polynucleotide sequence of the label probe system (e.g., L-2 can be complementary to a polynucleotide sequence of an amplification multimer, a preamplifier, a label probe, or the like). The label extender is preferably single-stranded. Label extenders designed in both directions are contemplated, i.e. a label extender in the 3′ to 5′ direction could just as easily be designed to bind in the reverse direction as depicted in the Figures. For instance, see FIGS. 7A and 7B for exemplary depictions of the various configurations which may be designed to be suitable for use in the presently disclosed invention.

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837, 3,850,752, 3,939,350, 3,996,345, 4,277,437, 4,275,149 and 4,366,241 (all of which are incorporated by reference in their entirety). Many labels are commercially available and can be used in the context of the invention.

A “label probe system” comprises one or more polynucleotides that collectively comprise a label and at least two polynucleotide sequences M-1, each of which is capable of hybridizing to a label extender. The label provides a signal, directly or indirectly. Polynucleotide sequence M-1 is typically complementary to sequence L-2 in the label extenders. The at least two polynucleotide sequences M-1 are optionally identical sequences or different sequences. The label probe system can include a plurality of label probes (e.g., a plurality of identical label probes) and an amplification multimer; it optionally also includes a preamplifier or the like, or optionally includes only label probes, for example.

An “amplification multimer” is a polynucleotide comprising a plurality of polynucleotide sequences M-2, typically (but not necessarily) identical polynucleotide sequences M-2. Polynucleotide sequence M-2 is complementary to a polynucleotide sequence in the label probe. The amplification multimer also includes at least one polynucleotide sequence that is capable of hybridizing to a label extender or to a nucleic acid that hybridizes to the label extender, e.g., a preamplifier. For example, the amplification multimer optionally includes at least one (and preferably at least two) polynucleotide sequence(s) M-1, optionally identical sequences M-1; polynucleotide sequence M-1 is typically complementary to polynucleotide sequence L-2 of the label extenders. Similarly, the amplification multimer optionally includes at least one polynucleotide sequence that is complementary to a polynucleotide sequence in a preamplifier. The amplification multimer can be, e.g., a linear or a branched nucleic acid. That is, the amplification multimer may be entirely comprised of a single contiguous chain of nucleic acids, or alternative a first chain possessing the sequence M-1 and additionally possessing one more sequences A-1 that are complementary to sequences A-2 on separate oligonucleotides which comprise one or more repeats of the sequence M-2. Thus, the amplification multimer may in fact be an assembly of multiple oligonucleotides comprising or consisting of a pre-amplifier possessing the M-2 sequence and one or more A-1 sequences; and one or more amplifier oligonucleotides possessing the sequence A-2 and one or more sequences M-2. Upon hybridization the structure may yield a tree-like geometrical shape comprising a single pre-amplifier, multiple amplifiers and attached to the amplifiers, multiple label probes which hybridize to site(s) M-2. As noted for all polynucleotides, the amplification multimer can include modified nucleotides and/or nonstandard internucleotide linkages as well as standard deoxyribonucleotides, ribonucleotides, and/or phosphodiester bonds. Suitable amplification multimers are described, for example, in U.S. Pat. Nos. 5,635,352, 5,124,246, 5,710,264 and 5,849,481 (all of which are incorporated herein by reference in their entirety).

A “label probe” or “LP” is a single-stranded polynucleotide that comprises a label (or optionally that is configured to bind to a label) that directly or indirectly provides a detectable signal. The label probe typically comprises a polynucleotide sequence that is complementary to the repeating polynucleotide sequence M-2 of the amplification multimer; however, if no amplification multimer is used in the bDNA assay, the label probe can, e.g., hybridize directly to a label extender.

A “preamplifier” is a nucleic acid that serves as an intermediate between one or more label extenders and amplifiers. Typically, the preamplifier is capable of hybridizing simultaneously to at least two label extenders and to a plurality of amplifiers.

A “microsphere” is a small spherical, or roughly spherical, particle. A microsphere typically has a diameter less than about 1000 micrometers (e.g., less than about 100 micrometers, optionally less than about 10 micrometers).

“Microparticles” include particles having a code, including sets of encoded microparticles. (See, for instance, U.S. Pat. Nos. 7,745,091 and 7,745,092 and U.S. patent application Ser. Nos. 11/521,115, 11/521,058, 11/521,153, and 12/215,607 and related applications, all of which are incorporated herein by reference in their entirety for all purposes). Such encoded microparticles may have a longest dimension of 50 microns, an outer surface substantially of glass and a spatial code that can be read with optical magnification. A microparticle may be cuboid in shape and elongated along the Y direction in the Cartesian coordinate. The cross-sections perpendicular to the length of the microparticle may have substantially the same topological shape—such as square shape. Microparticles may have a set of segments and gaps intervening the segments in parallel along the axis of the longest dimension if the microparticle is rectangular. Specifically, segments with different lengths (the dimension along the length of the microparticle, e.g. along the Y direction) may represent different coding elements; whereas gaps preferably have the same length for differentiating the segments during detection of the microparticles. The segments of the microparticle may be fully enclosed within the microparticle, i.e. completely encapsulated by a surrounding outer layer which may be silicon/glass. As an alternative feature, the segments can be arranged such that the geometric centers of the segments are aligned to the geometric central axis of the elongated microparticle. A particular sequence of segments and gaps thereby represent a code within each microparticle. The codes may be derived from a pre-determined coding scheme thereby allowing identification of the microparticle. The microparticles may additionally have various structural aberrations, such as tags or tabs, on one or more ends, thus allowing for a two-fold or more increase in code space. The microparticles may also be present as a “bi-particle” wherein the microparticle actually comprises two or more particles stuck together, i.e. missing the last etching step so as to allow two particles to remain attached together with an intervening material between them comprised of material consistent with the coating present on the rest of the microparticle. (See, for instance, U.S. patent application Ser. No. 12/779,413, filed May 13, 2010, incorporated herein by reference in its entirety for all purposes). The microparticle may have covalently attached thereto various biological components including antigens, antibodies, nucleic acids and/or combinations and/or mixtures thereof. The components covalently attached to the microparticle may optionally be either randomly dispersed throughout the surface of the barcode or spatially localized to specific regions of the surface of the barcode, i.e. on the edge or short-side of the barcode, on the broader flat surface of the barcode, or combinations such as antibodies located on the surface of one end of the barcode and, for example, probes located at the other opposite end of the same microparticle. The benefits and capabilities of various coverage combinations and arrangements on the microparticles will be apparent to one of skill in the art.

A “microorganism” is an organism of microscopic or submicroscopic size. Examples include, but are not limited to, bacteria, fungi, yeast, protozoans, microscopic algae (e.g., unicellular algae), viruses (which are typically included in this category although they are incapable of growth and reproduction outside of host cells), subviral agents, viroids, and mycoplasma.

A first polynucleotide sequence that is located “5′ of” a second polynucleotide sequence on a nucleic acid strand is positioned closer to the 5′ terminus of the strand than is the second polynucleotide sequence. Similarly, a first polynucleotide sequence that is located “3′ of” a second polynucleotide sequence on a nucleic acid strand is positioned closer to the 3′ terminus of the strand than is the second polynucleotide sequence.

A variety of additional terms are defined or otherwise characterized herein.

### DETAILED DESCRIPTION

The present invention provides methods, compositions, and kits for detection of various types of nucleic acids and proteins found inside or on the external surface of cells, particularly multiplex detection of such nucleic acids and proteins. As will be shown in more detail below, the disclosed methodologies and compositions are highly adaptable to many applications including identification and quantitation of specific cells and/or cell types as well as determination of cell type and/or disease state, and the like.

A. Labeling of Cells with Microparticles and Detection Thereof

Generally, bDNA assays are aimed at methods of identification of two or more nucleic acids of interest. The nucleic acids may or may not be methylated. In such assays, a sample, a pooled population of particles (or microparticles, or encoded microparticles), and two or more subsets of n target capture probes, wherein n is at least two, are used. The sample comprises or is suspected of comprising the nucleic acids of interest. The pooled population of particles includes two or more subsets of particles. The particles in each subset have associated therewith a different capture probes. Each subset of n capture extenders is capable of hybridizing to one of the nucleic acids of interest, and the capture extenders in each subset are capable of hybridizing to one of the capture probes and thereby associating each subset of n target capture probes with a selected subset of the particles. Preferably, a plurality of the particles in each subset is distinguishable from a plurality of the particles in every other subset. (Typically, substantially all of the particles in each subset are distinguishable from substantially all of the particles in every other subset.) Each nucleic acid of interest can thus, by hybridizing to its corresponding subset of n capture extenders which are in turn hybridized to a corresponding capture probes, be associated with an identifiable subset of the particles. Alternatively, the particles in the various subsets need not be distinguishable from each other (for example, in embodiments in which any nucleic acid of interest present is to be isolated, amplified, and/or detected, without regard to its identity, following its capture on the particles.) Such methods of using capture probes (CP) and capture extenders (CE) to bind a target nucleic acid sequence to a particle are well known in the art and the subject of many US patents, as discussed and disclosed in more detail, below.

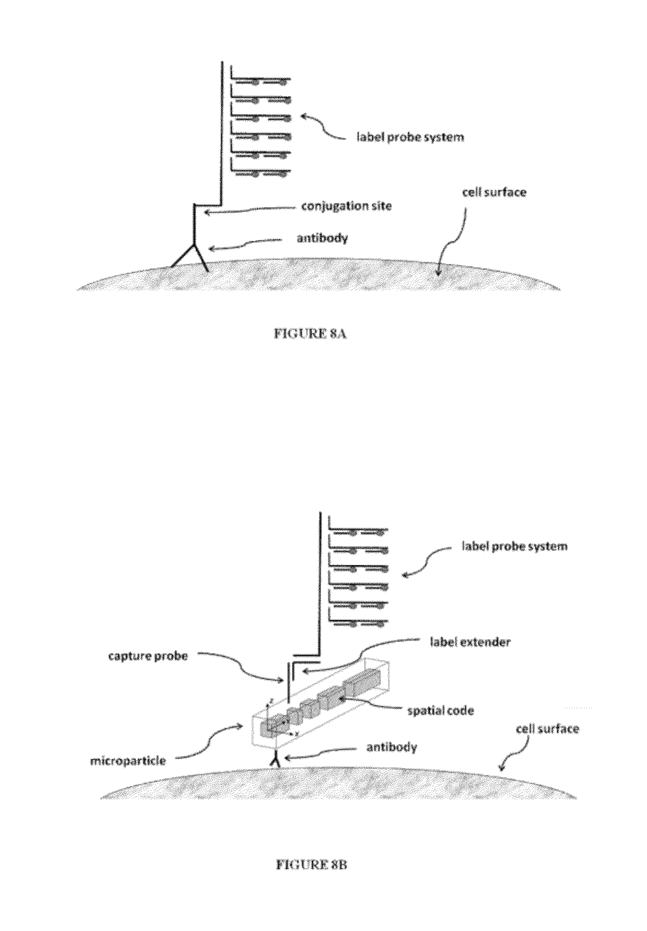

These general bDNA assay methods are highly versatile and have now been found to be very useful in marking and tagging cells using microparticles, such as encoded microparticles. In general, the assay may proceed as follows. An encoded microparticle has attached thereto a specific set of nucleic acids used for label amplification and identification purposes, i.e. to maintain identity of the microparticle and to detect the microparticle. Other parts of the microparticle will have attached either antigens, antibodies, or mixtures and combinations of both (see, for instance, FIG. 8B). Thus, an encoded microparticle having a readable spatial code therein, will have attached to it antibodies and/or antigens as well as nucleotides or probes. The choice of antibody or antigen depends on which cells are to be targeted or labeled with the microparticle. For instance, there exists a myriad different antibodies known to specifically bind to external membrane proteins of specific types of cells, i.e. antibodies against MHC class II molecules which target only cells expressing MHC class II receptors, etc. If it is desired to label only these types of cells, that type of antibody is affixed, either covalently or non-covalently, to the microparticles. Likewise, antigens known to bind to specific cellular receptors can be selected to affix to the microparticle. Incubation of the microparticle with a mixture of cells will then result in binding of the microparticle to the cell, via the antibody or antigen affixed to the microparticle. The microparticle may then be read directly to determine its spatial code, or the solution of cells and microparticles may then be incubated with the bDNA label probe system to amplify a signal targeted to specific nucleotide target sequences also affixed to the microparticles, thereby applying an amplified signal to specific subsets of the microparticles. Various optional washing steps may be employed to remove unbound particles before, during and/or after binding of particles to cells and/or binding of the bDNA label probe system to the particles.

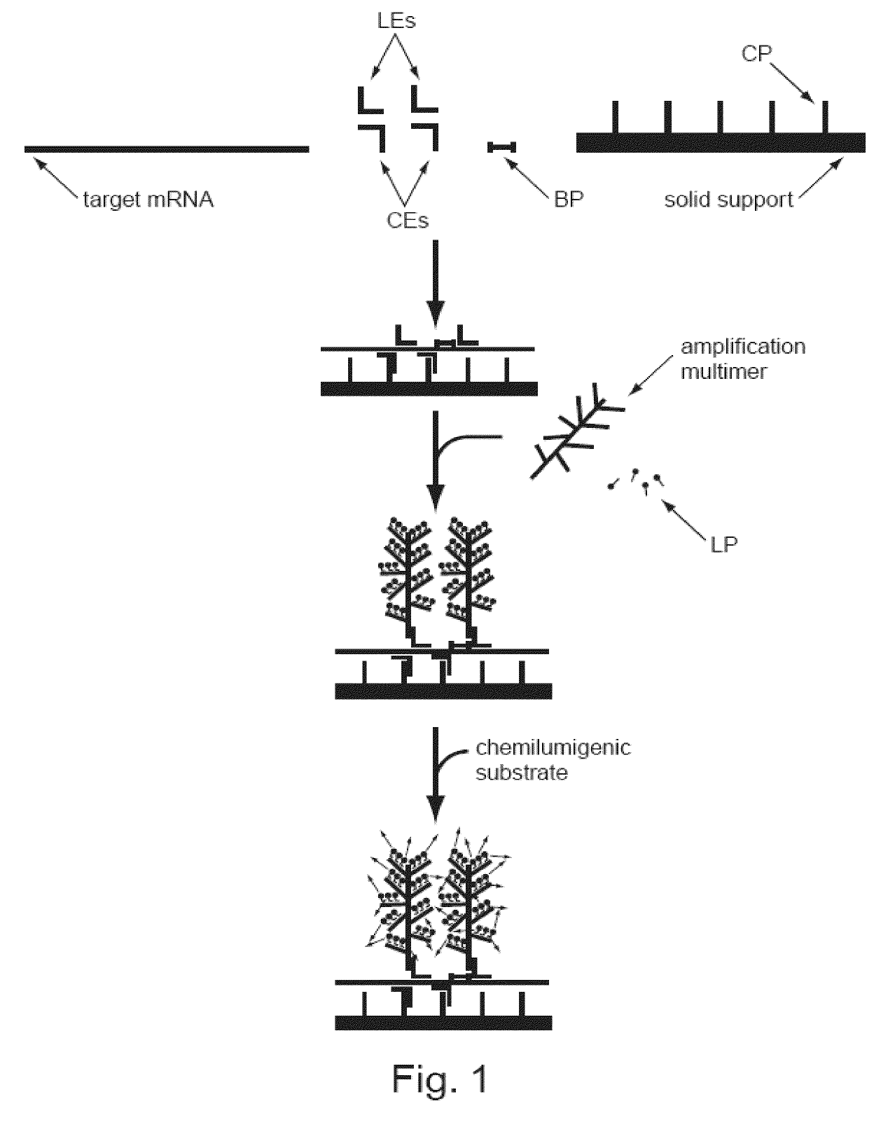

Below is provided a brief summarization of bDNA technology and how it works. This technology enables rapid and highly specific signal amplification as applied to the cell labeling methodologies and compositions described in brief above. There are at least two general classes of bDNA technology which may be employed in the labeling and detection of microparticles bound to cells. One general class utilizes a direct approach of binding label extender probes (LE) directly to capture probes affixed to the microparticle with no intermediary target nucleic acid or capture extender (CE) probes. (See, FIG. 1). The amplifiers or preamplifiers and amplifiers along with label probes may then be assembled directly onto the LEs bound to the CPs. As is explained in more detail below, the LEs provide added specificity in targeting only those sequences complementary to their CP sequences. As an alternative, even simpler, embodiment, it is possible to bind amplifiers directly to the capture probes on the microparticles. In a second general class of embodiments, a more standard bDNA labeling architecture is employed as in FIG. 1. In this second embodiment, the target nucleic acid can be any sequence desirable and optimized for the purpose of specifically binding to LEs and the remainder of the label probe system depicted in FIG. 1. These two approaches are not the only possible approaches but are set forth here as exemplary embodiments allowing the assembly of the bDNA label probe system onto the microparticle which is itself also bound to the cell or various cell types the user wishes to detect. Various different target nucleic acid sequences may be used which then correspond to specific CEs and thereby specific subpopulations of particles and in turn specific cells or cell types, allowing differential multi-colored labeling and multiplexing of the assay. Similarly, with respect to the first exemplary embodiment, different LEs may be used having specificity for different amplifier/preamplifier combinations which in turn only bind specific types of label probes (due to the specific complementary sequences encoded therein) and specific labels of different types again allowing for multiplexing, use of different labels and even mixing and matching of label colors which could then possibly be detected using a two-channel or multi-channel detector. Finally, in yet another embodiment, no bDNA label probe system is needed. In this third embodiment, the spatial codes in the microparticles themselves are simply detected with no use of labels or nucleic-acid based signal amplification.

In one embodiment of the following methodologies and compositions, a capture probe (CP) residing on a microparticle of interest may be detected through signal amplification using bDNA. Branched-chain DNA (bDNA) signal amplification technology has been used, e.g., to detect and quantify mRNA transcripts in cell lines and to determine viral loads in blood. (See, for instance, Player et al. (2001) “Single-copy gene detection using branched DNA (bDNA) in situ hybridization,” J. Histochem. Cytochem., 49:603-611, Van Cleve et al., Mol. Cell. Probes, (1998) 12:243-247, and U.S. Pat. No. 7,033,758, each of which is incorporated herein by reference in their entirety for all purposes). The bDNA assay is essentially a sandwich nucleic acid hybridization procedure that enables direct measurement of target nucleic acid. Several advantages of the bDNA amplification technology distinguishes it from other DNA/RNA amplification technologies, including linear amplification, good sensitivity and dynamic range, great precision/specificity and accuracy, simple sample preparation procedure, and reduced sample-to-sample variation.

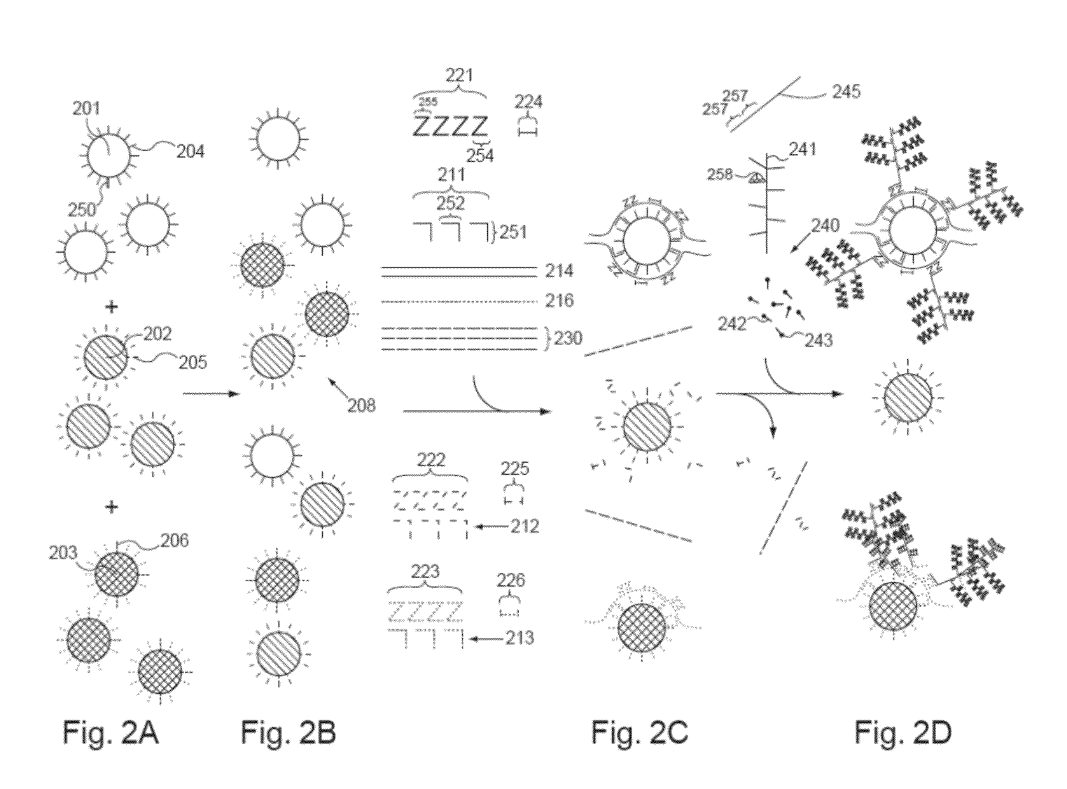

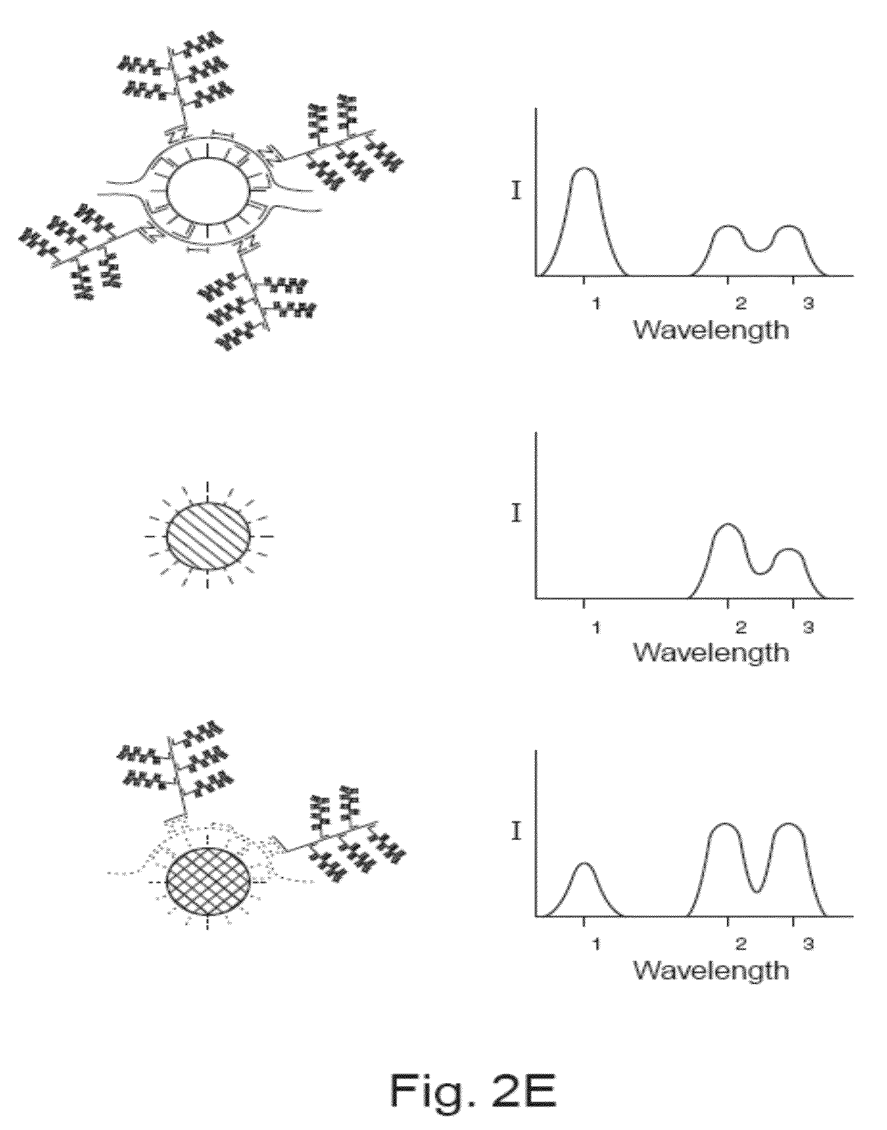

In brief, in a typical bDNA assay of the type useful in the present embodiments, probes called Label Extenders (LEs) are used to hybridize to different sequences on the target nucleic acid and to sequences on an amplification multimer. For instance, please refer to the top left corner of FIG. 2E where there is depicted a microparticle circle on which are various capture probes, bound to capture extenders, which are then bound to a target nucleic acid which is in turn bound to Label Extender Probes (depicted as a small “ZZ” shape around the circle). Label Extender Probes are then bound to amplifiers which then can bind Label Probes. Additionally, Blocking Probes (BPs), which hybridize to regions of the target nucleic acid not occupied by LEs, are often used to reduce non-specific target probe binding. A probe set for a given nucleic acid target in the present embodiment thus consists of LEs, and optionally BPs for the target nucleic acid as well as amplification multimers, etc. The LEs and BPs are complementary to nonoverlapping sequences in the target nucleic acid, and are typically, but not necessarily, contiguous. Again, in this embodiment, the sequence of the target nucleic acid is completely known and designed for optimum efficiency and specificity of assembly of the bDNA label probe system as depicted in FIGS. 1 and 2. The main reason for assembling these various probes to the microparticle is for detection purposes, not to bind to de novo nucleic acids found in the sample solution.

Signal amplification begins with the binding of the LEs to the target nucleic acid or to Capture Probes. An amplification multimer is then typically hybridized to the LEs. The amplification multimer has multiple copies of a sequence that is complementary to a label probe (it is worth noting that the amplification multimer is typically, but not necessarily, a branched-chain nucleic acid; for example, the amplification multimer can be a branched, forked, or comb-like nucleic acid or a linear nucleic acid). A label, for example, alkaline phosphatase (or any other label as further described below), is associated with each label probe. (The label may be noncovalently bound to the label probes.) In the final step, labeled complexes are detected, e.g., by the alkaline phosphatase-mediated degradation of a chemilumigenic substrate, e.g., dioxetane. Luminescence is reported as relative light unit (RLUs). The amount of chemiluminescence is proportional to the level of mRNA expressed from the target gene.

In the preceding example, the amplification multimer and the label probes comprise a label probe system. In another example, the label probe system also comprises a preamplifier, e.g., as described in U.S. Pat. Nos. 5,635,352 and 5,681,697 (both of which are incorporated by reference in their entirety), which further amplifies the signal from a single target nucleic acid. In yet another example, the label extenders hybridize directly to the label probes and no amplification multimer or preamplifier is used, so the signal from a single target nucleic acid is only amplified by the number of distinct label extenders that hybridize to that target nucleic acid.

Basic bDNA assays have been well described. (See, e.g., U.S. Pat. No. 4,868,105 to Urdea et al. entitled “Solution phase nucleic acid sandwich assay”; U.S. Pat. No. 5,635,352 to Urdea et al. entitled “Solution phase nucleic acid sandwich assays having reduced background noise”; U.S. Pat. No. 5,681,697 to Urdea et al. entitled “Solution phase nucleic acid sandwich assays having reduced background noise and kits therefor”; U.S. Pat. No. 5,124,246 to Urdea et al. entitled “Nucleic acid multimers and amplified nucleic acid hybridization assays using same”; U.S. Pat. No. 5,624,802 to Urdea et al. entitled “Nucleic acid multimers and amplified nucleic acid hybridization assays using same”; U.S. Pat. No. 5,849,481 to Urdea et al. entitled “Nucleic acid hybridization assays employing large comb-type branched polynucleotides”; U.S. Pat. No. 5,710,264 to Urdea et al. entitled “Large comb type branched polynucleotides”; U.S. Pat. No. 5,594,118 to Urdea and Horn entitled “Modified N-4 nucleotides for use in amplified nucleic acid hybridization assays”; U.S. Pat. No. 5,093,232 to Urdea and Horn entitled “Nucleic acid probes”; U.S. Pat. No. 4,910,300 to Urdea and Horn entitled “Method for making nucleic acid probes”; U.S. Pat. No. 5,359,100; U.S. Pat. No. 5,571,670; U.S. Pat. No. 5,614,362; U.S. Pat. No. 6,235,465; U.S. Pat. No. 5,712,383; U.S. Pat. No. 5,747,244; U.S. Pat. No. 6,232,462; U.S. Pat. No. 5,681,702; U.S. Pat. No. 5,780,610; U.S. Pat. No. 5,780,227 to Sheridan et al. entitled “Oligonucleotide probe conjugated to a purified hydrophilic alkaline phosphatase and uses thereof”; U.S. patent application Publication No. US2002172950 by Kenny et al. entitled “Highly sensitive gene detection and localization using in situ branched-DNA hybridization”; Wang et al. (1997) “Regulation of insulin preRNA splicing by glucose” Proc Nat Acad Sci USA 94:4360-4365; Collins et al. (1998) “Branched DNA (bDNA) technology for direct quantification of nucleic acids: Design and performance” in Gene Quantification, F Ferre, ed.; and Wilber and Urdea (1998) “Quantification of HCV RNA in clinical specimens by branched DNA (bDNA) technology” Methods in Molecular Medicine: Hepatitis C 19:71-78, all of which are incorporated herein by reference in their entirety). In addition, kits for performing basic bDNA assays (QUANTIGENE® kits, comprising instructions and reagents such as amplification multimers, alkaline phosphatase labeled label probes, chemilumigenic substrate, capture probes immobilized on a solid support, and the like) are commercially available, e.g., from Affymetrix, Inc. (on the world wide web at the Affymetrix website). General protocols and user's guides on how the QUANTIGENE® system works and explanation of kits and components may be found at the Affymetrix website (see, www.panomics.com/index.php?id=product_{—}1#product_lit_{—}1). Specifically, user's manual, “QUANTIGENE® 2.0 Reagent System User Manual,” (2007, 32 pages) provided at the Affymetrix website is incorporated herein by reference in its entirety for all purposes. Software for designing probe sets for a given target nucleic acid (i.e., for designing the regions of the LEs, and optionally BPs, that are complementary to the target) is also commercially available (e.g., see Bushnell et al. (1999) “ProbeDesigner: for the design of probe sets for branched DNA (bDNA) signal amplification assays,” Bioinformaticsm 15:348-55, incorporated herein by reference).

Among other aspects, the present invention provides multiplex bDNA assays that can be used for simultaneous detection of two or more target nucleic acids, thereby allowing for differential labeling, using the label probe system, of each microparticle population with a different spatial code, and/or subsets thereof. Similarly, one aspect of the present invention provides bDNA assays, singleplex or multiplex, that possess reduced background from nonspecific hybridization events.

Among other aspects, the present invention provides a multiplex bDNA assay that can be used for simultaneous detection of two or more target nucleic acids, and thereby labeling of two or more microparticles, which thereby provides for the barcoding or labeling of two or more different cell types.

In general, in the assays of the invention, as in the first embodiment discussed above, two or more label extenders are used to hybridize to the Capture Probes and simultaneously to capture a single component of the label probe system (e.g., a preamplifier or amplification multimer). The assay temperature and the stability of the complex between a single LE and the component of the label probe system (e.g., the preamplifier or amplification multimer) can be controlled such that binding of a single LE to the component is not sufficient to stably associate the component with a nucleic acid to which the LE is bound, whereas simultaneous binding of two or more LEs to the component can capture it to the nucleic acid. Requiring such cooperative hybridization of multiple LEs for association of the label probe system with the Capture Probe (or target nucleic acid as in FIG. 1, whichever embodiment is being used) results in high specificity and low background from cross-hybridization of the LEs with other, non-target nucleic acids.

For an assay to achieve high specificity and sensitivity, it preferably has a low background, resulting, e.g., from minimal cross-hybridization. Such low background and minimal cross-hybridization are typically substantially more difficult to achieve in a multiplex assay than a single-plex assay, because the number of potential nonspecific interactions are greatly increased in a multiplex assay due to the increased number of probes used in the assay (e.g., the greater number of LEs). Requiring multiple simultaneous LE-label probe system component interactions for the capture of the label probe system to a target nucleic acid (or capture probe) minimizes the chance that nonspecific capture will occur, even when some nonspecific interactions do occur. This reduction in background through minimization of undesirable cross-hybridization events thus facilitates multiplex detection of the nucleic acids of interest.

The methods of the invention can be used, for example, for multiplex detection of two or more cells or cell types simultaneously, from even complex samples, without requiring prior purification of the cell.

Thus, one general class of embodiments includes methods of labeling two or more target nucleic acids bound to microparticles, which in turn are bound to specific target cells or cell types. In one embodiment of the method, a sample comprising or suspected of comprising the nucleic acids of interest, i.e. before or after optional washing steps, two or more subsets of m label extenders, wherein m is at least two, and a label probe system are provided. Each subset of m label extenders is capable of hybridizing to one of the target nucleic acids. The label probe system comprises a label, and a component of the label probe system is capable of hybridizing simultaneously to at least two of the m label extenders in a subset. Each capture probe attached to the microparticle is hybridized to its corresponding subset of m label extenders, and the label probe system is hybridized to the m label extenders. The presence or absence of the label on the solid support is then detected. Since the label is associated with the nucleic acid(s) of interest via hybridization of the label extenders and label probe system, the presence or absence of the label on the solid support is correlated with the presence or absence of the nucleic acid(s) of interest on the solid support and thus in the original sample.

The population of particles or microparticles utilized in the presently described assays may comprise two or more subsets of particles, and a plurality of the particles in each subset is distinguishable from a plurality of the particles in every other subset. (Typically, substantially all of the particles in each subset are distinguishable from substantially all of the particles in every other subset.) Typically, in this class of embodiments, at least a portion of the particles from each subset are identified and the presence or absence of the label on those particles is detected. Since a correlation exists between a particular subset of particles and a particular cell of interest, which subsets of particles are labeled indicates which of cells of interest were present in the sample.

Essentially any suitable particles, e.g., particles having distinguishable characteristics and to which capture probes can be attached, can be used. For example, in one preferred class of embodiments, the particles are microspheres or microparticles or encoded microparticles. The microspheres of each subset can be distinguishable from those of the other subsets, e.g., on the basis of their fluorescent emission spectrum, their diameter, or a combination thereof. For example, the microspheres of each subset can be labeled with a unique fluorescent dye or mixture of such dyes, quantum dots with distinguishable emission spectra, and/or the like. As another example, the particles of each subset can be identified by an optical barcode, unique to that subset, present on or in the particles or microparticles, as in encoded microparticles, for instance.

For a given target nucleic acid, or capture probe, the corresponding label extenders are preferably complementary to physically distinct, nonoverlapping sequences in the target nucleic acid or capture probe, which are preferably, but not necessarily, contiguous. The Tₘs of the individual label extender-target nucleic acid/capture probe complexes are preferably greater than the hybridization temperature, e.g., by 5° C. or 10° C. or preferably by 15° C. or more, such that these complexes are stable at the hybridization temperature. Potential label extender sequences are optionally examined for possible interactions with non-corresponding nucleic acids, repetitive sequences (such as polyC or polyT, for example) and/or any relevant sequences which may also be present in the assay, for example; sequences expected to cross-hybridize with undesired nucleic acids are typically not selected for use in the label extenders. Examination can be, e.g., visual (e.g., visual examination for complementarity), computational (e.g., computation and comparison of percent sequence identity and/or binding free energies; for example, sequence comparisons can be performed using BLAST software publicly available through the National Center for Biotechnology Information on the world wide web at ncbi.nlm.nih.gov), and/or experimental (e.g., cross-hybridization experiments).

The methods are useful for multiplex detection of nucleic acids, optionally highly multiplex detection. Thus, the two or more target nucleic acids or capture probes optionally comprise five or more, 10 or more, 20 or more, 30 or more, 40 or more, 50 or more, or even 100 or more target nucleic acids, while the two or more subsets of m label extenders comprise five or more, 10 or more, 20 or more, 30 or more, 40 or more, 50 or more, or even 100 or more subsets of m label extenders. In embodiments in which label extenders, particulate solid supports, and/or spatially addressable solid support are used, a like number of subsets of label extenders, subsets of particles, and/or selected positions on the solid support are provided.

The label probe system optionally includes an amplification multimer and a plurality of label probes, wherein the amplification multimer is capable of hybridizing to the label extenders and to a plurality of label probes. In another aspect, the label probe system includes a preamplifier, a plurality of amplification multimers, and a plurality of label probes, wherein the preamplifier hybridizes to the label extenders, and the amplification multimers hybridize to the preamplifier and to the plurality of label probes (see FIG. 1). In one class of embodiments, the label probe comprises the label, e.g., a covalently attached label. In other embodiments, the label probe is configured to bind a label; for example, a biotinylated label probe can bind to a streptavidin-associated label.

The label can be essentially any convenient label that directly or indirectly provides a detectable signal. In one aspect, the label is a fluorescent label (e.g., a fluorophore or quantum dot). Detecting the presence of the label on the particles thus comprises detecting a fluorescent signal from the label. In embodiments in which the solid support comprises particles, fluorescent emission by the label is typically distinguishable from any fluorescent emission by the particles, e.g., microspheres, and many suitable fluorescent label-fluorescent microsphere combinations are possible. As other examples, the label can be a luminescent label, a light-scattering label (e.g., colloidal gold particles), or an enzyme (e.g., HRP). Various labels are known in the art, such as Alexa Fluor Dyes (Life Technologies, Inc., California, USA, available in a wide variety of wavelengths, see for instance, Panchuk, et al., J. Hist. Cyto., 47:1179-1188, 1999), biotin-based dyes, digoxigenin, AttoPhos (JBL Scientific, Inc., California, USA, available in a variety of wavelengths, see for instance, Cano et al., Biotechniques, 12(2):264-269, 1992), ATTO dyes (Sigma-Aldrich, St. Louis, Mo.), or any other suitable label. Furthermore, lanthanide labels may also be employed, such as those provided by DVS Sciences, Inc. (Sunnyvale, Calif.). (See, Ornatsky et al., “Study of Cell Antigens and Intracellular DNA by Identification of Element-Containing Labels and Metallointercalators Using inductively Coupled Plasma Mass Spectrometry,” Anal. Chem., 80:2539, 2008, incorporated herein by reference in its entirety for all purposes).

As noted above, a component of the label probe system is capable of hybridizing simultaneously to at least two of the m label extenders in a subset. Typically, the component of the label probe system that hybridizes to the two or more label extenders is an amplification multimer or preamplifier. Preferably, binding of a single label extender to the component of the label probe system (e.g., the amplification multimer or preamplifier) is insufficient to capture the label probe system to the nucleic acid of interest to which the label extender binds. Thus, in one aspect, the label probe system comprises an amplification multimer or preamplifier, which amplification multimer or preamplifier is capable of hybridizing to the at least two label extenders, and the label probe system (or the component thereof) is hybridized to the m label extenders at a hybridization temperature, which hybridization temperature is greater than a melting temperature Tₘ of a complex between each individual label extender and the amplification multimer or preamplifier. The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the T. It is worth noting that the hybridization temperature can be the same or different than the temperature at which the label extenders and optional capture extenders are hybridized to the nucleic acids of interest.

Each label extender typically includes a polynucleotide sequence L-1 that is complementary to a polynucleotide sequence in the corresponding nucleic acid of interest and a polynucleotide sequence L-2 that is complementary to a polynucleotide sequence in the component of the label probe system (e.g., the preamplifier or amplification multimer). It will be evident that the amount of overlap between each individual label extender and the component of the label probe system (i.e., the length of L-2 and M-1) affects the Tₘ of the complex between the label extender and the component, as does, e.g., the GC base content of sequences L-2 and M-1. Optionally, all the label extenders have the same length sequence L-2 and/or identical polynucleotide sequences L-2. Alternatively, different label extenders can have different length and/or sequence polynucleotide sequences L-2. It will also be evident that the number of label extenders required for stable capture of the component to the nucleic acid of interest depends, in part, on the amount of overlap between the label extenders and the component (i.e., the length of L-2 and M-1).

Stable capture of the component of the label probe system by the at least two label extenders, e.g., while minimizing capture of extraneous nucleic acids, can be achieved, for example, by balancing the number of label extenders that bind to the component, the amount of overlap between the label extenders and the component (the length of L-2 and M-1), and/or the stringency of the conditions under which the label extenders and the component are hybridized. For instance, when detecting a large message RNA of several hundred base pairs or less, any number of label extenders may be used, such as, for instance, 1-30 pairs of label extender probes, or 2-28 pairs of label extender probes, or 3-25 pairs of label extender probes, or 4-20 pairs of label extender probes, or a number of label extender probe pairs which is suitable to specifically attach the label probe system to the target with the desired affinity.

As noted above, while some embodiments generally utilize two label extender probes to hybridize to each pre-amplifier, it is possible in other embodiments to design systems in which three label extender probes hybridize to a single target and single pre-amplifier probe, or even four label extender probes per pre-amplifier. Further, it is possible to use only a single label extender probe, in concert with a single capture probe, to detect the target (as in the second embodiment as mentioned above). Alternatively, if performing the assay in situ, for example, or in other suitable conditions, a single pair of label extender probes may be designed to contain the entire complement to the target nucleic acid or capture probe sequence (half of which would be encoded in the L-1 sequence of a first label extender probe, and the other half of which would be encoded in the second L-1 sequence of the second label extender probe).

Appropriate combinations of the amount of complementarity between the label extenders and the component of the label probe system, number of label extenders binding to the component, and stringency of hybridization, can be determined experimentally by one of skill in the art. For example, a particular number of label extenders and a particular set of hybridization conditions can be selected, while the number of nucleotides of complementarity between the label extenders and the component is varied until hybridization of the label extenders to a nucleic acid captures the component to the nucleic acid while hybridization of a single label extender does not efficiently capture the component. Stringency can be controlled, for example, by controlling the formamide concentration, chaotropic salt concentration, salt concentration, pH, organic solvent content, and/or hybridization temperature.

As noted, the Tₘ of any nucleic acid duplex can be directly measured, using techniques well known in the art. For example, a thermal denaturation curve can be obtained for the duplex, the midpoint of which corresponds to the T. It will be evident that such denaturation curves can be obtained under conditions having essentially any relevant pH, salt concentration, solvent content, and/or the like.

The Tₘ for a particular duplex (e.g., an approximate Tₘ) can also be calculated. For example, the Tₘ for an oligonucleotide-target duplex can be estimated using the following algorithm, which incorporates nearest neighbor thermodynamic parameters: Tm (Kelvin)=ΔH°/(ΔS°+R ln Cₜ), where the changes in standard enthalpy (ΔH°) and entropy (ΔS°) are calculated from nearest neighbor thermodynamic parameters (see, e.g., SantaLucia (1998) “A unified view of polymer, dumbbell, and oligonucleotide DNA nearest-neighbor thermodynamics” Proc. Natl. Acad. Sci. USA 95:1460-1465, Sugimoto et al. (1996) “Improved thermodynamic parameters and helix initiation factor to predict stability of DNA duplexes” Nucleic Acids Research 24: 4501-4505, Sugimoto et al. (1995) “Thermodynamic parameters to predict stability of RNA/DNA hybrid duplexes” Biochemistry 34:11211-11216, and et al. (1998) “Thermodynamic parameters for an expanded nearest-neighbor model for formation of RNA duplexes with Watson-Crick base pairs” Biochemistry 37: 14719-14735), R is the ideal gas constant (1.987 cal·K⁻¹mole⁻¹), and Cₜ is the molar concentration of the oligonucleotide. The calculated Tₘ is optionally corrected for salt concentration, e.g., Na⁺ concentration, using the formula:

1/Tₘ(Na⁺)=1/Tₘ(1M)+(4.29f(G·C)−3.95)×10⁻⁵ ln [Na⁺]+9.40×10⁻⁶ ln² [Na⁺]

(See, e.g., Owczarzy et al. (2004) “Effects of Sodium Ions on DNA Duplex Oligomers: Improved Predictions of Melting Temperatures,” Biochemistry, 43:3537-3554 for further details, incorporated herein by reference in its entirety). A Web calculator for estimating Tₘ using the above algorithms is available on the Internet at scitools.idtdna.com/analyzer/oligocalc.asp. Other algorithms for calculating Tₘ are known in the art and are optionally applied to the present invention.

Typically, the component of the label probe system (e.g., the amplification multimer or preamplifier) is capable of hybridizing simultaneously to two of the m label extenders in a subset, although it optionally hybridizes to three, four, or more of the label extenders. In one class of embodiments, e.g., embodiments in which two (or more) label extenders bind to the component of the label probe system, sequence L-2 is 20 nucleotides or less in length. For example, L-2 can be between 9 and 17 nucleotides in length, e.g., between 12 and 15 nucleotides in length, between 13 and 15 nucleotides in length, or between 13 and 14 nucleotides in length. As noted, m is at least two, and can be at least three, at least five, at least 10, or more. Additionally, “m” can be the same or different from subset to subset of label extenders.

The label extenders can be configured in any of a variety ways. For example, the two label extenders that hybridize to the component of the label probe system can assume a cruciform arrangement, with one label extender having L-1 5′ of L-2 and the other label extender having L-1 3′ of L-2. Thus, in one class of embodiments, the at least two label extenders (e.g., the m label extenders in a subset) each have L-1 5′ of L-2 or each have L-1 3′ of L-2. For example, L-1, which hybridizes to the nucleic acid of interest, can be at the 5′ end of each label extender, while L-2, which hybridizes to the component of the label probe system, is at the 3′ end of each label extender (or vice versa). L-1 and L-2 are optionally separated by additional sequence. In one exemplary embodiment, L-1 is located at the 5′ end of the label extender and is about 20-30 nucleotides in length, L-2 is located at the 3′ end of the label extender and is about 13-14 nucleotides in length, and L-1 and L-2 are separated by a spacer (e.g., 5 Ts).

A label extender, target nucleic acid, preamplifier, amplification multimer, label probe and/or capture probe optionally comprise at least one non-natural nucleotide. For example, a label extender and the component of the label probe system (e.g., the amplification multimer or preamplifier) optionally comprise, at complementary positions, at least one pair of non-natural nucleotides that base pair with each other but that do not Watson-Crick base pair with the bases typical to biological DNA or RNA (i.e., A, C, G, T, or U). Examples of nonnatural nucleotides include, but are not limited to, Locked Nucleic Acid nucleotides (LNA, an example of which is available from Exiqon A/S, (www.) exiqon.com; see, e.g., SantaLucia Jr. (1998) Proc. Natl. Acad. Sci., 95:1460-1465) and isoG, isoC, and other nucleotides used in the AEGIS system (Artificially Expanded Genetic Information System, available from EraGen Biosciences, (www.) eragen.com; see, e.g., U.S. Pat. No. 6,001,983, U.S. Pat. No. 6,037,120, and U.S. Pat. No. 6,140,496, see also FIGS. 4A and 4B). Use of such non-natural base pairs (e.g., isoG-isoC base pairs) in the probes can, for example, reduce background and/or simplify probe design by decreasing cross hybridization, or it can permit use of shorter probes (e.g., shorter sequences L-2 and M-1) when the non-natural base pairs have higher binding affinities than do natural base pairs.

At any of various steps, cells not captured on the particles are optionally separated from the bound cells. For example, after the label extenders, amplifiers and/or pre-amplifiers and blocking probes are hybridized, the cells bound with particles are optionally washed to remove non-bound cells, particles and probes; after the label extenders and amplification multimer are hybridized, the cells are optionally washed to remove unbound amplification multimer; and/or after the label probes are hybridized to the amplification multimer, the cells are optionally washed to remove unbound label probe prior to detection of the label.

The methods can be used to detect the presence of specific cells and cell types of interest in essentially any type of sample. For example, the sample can be derived from an animal, a human, a plant, a cultured cell, a virus, a bacterium, a pathogen, and/or a microorganism. The sample optionally includes a bodily fluid (including, but not limited to, blood, serum, saliva, urine, sputum, or spinal fluid), and/or a conditioned culture medium, and is optionally derived from a tissue (e.g., a tissue homogenate), a biopsy, and/or a tumor. Similarly, any of the nucleic acids and probes can be essentially any desired nucleic acids (e.g., DNA, methylated DNA, RNA, mRNA, rRNA, miRNA, siRNA, etc.) and may further comprise one or more stretches of one or more known nucleic acid analogs. Likewise any number of nucleic acids found in the label extenders and/or label probes may also be comprised of one or more stretches of one or more known nucleic acid analogs.

An exemplary embodiment of traditional bDNA assays is schematically illustrated in FIG. 2. Panel A illustrates three subsets of label extenders (221, 222, and 223 for nucleic acids 214, 215, and 216, respectively) and three subsets of blocking probes (224, 225, and 226 for nucleic acids 214, 215, and 216, respectively) are also provided. Each label extender includes sequences L-1 (254, complementary to a sequence in the corresponding nucleic acid of interest) and L-2 (255, complementary to M-1). Non-target nucleic acids 230 are also present in the sample.

Subsets of label extenders 221 and 223 are hybridized to nucleic acids 214 and 216, respectively. In addition, in embodiments which utilize a target nucleic acid which is captured by the capture probes and then to which is bound label extender probes, nucleic acids 214 and 216 are hybridized to their corresponding subset of capture extenders (211 and 213, respectively), and the capture extenders are hybridized to the corresponding capture probes (204 and 206, respectively), capturing nucleic acids 214 and 216 on microspheres 201 and 203, respectively (Panel C). Materials not bound to the microspheres (e.g., capture extenders 212, nucleic acids 230, etc.) are separated from the microspheres by washing. Label probe system 240 including preamplifier 245 (which includes two sequences M-1 257), amplification multimer 241 (which includes sequences M-2 258), and label probe 242 (which contains label 243) is provided. Each preamplifier 245 is hybridized to two label extenders, amplification multimers 241 are hybridized to the preamplifier, and label probes 242 are hybridized to the amplification multimers (Panel D). Materials not captured on the microspheres are optionally removed by washing the microspheres. Microspheres from each subset are identified, e.g., by their fluorescent emission spectrum (λ₂ and λ₃, Panel E), and the presence or absence of the label on each subset of microspheres is detected (λ₁, Panel E). Since each nucleic acid of interest is associated with a distinct subset of microspheres, the presence of the label on a given subset of microspheres correlates with the presence of the corresponding nucleic acid in the original sample. It is noted again that this is merely one embodiment and that other simpler embodiments may be employed which do not use a target nucleic acid intermediary and simply hybridize label extender probes directly to the capture probe for assembly of the label probe system.

As depicted in FIG. 2, all of the label extenders in all of the subsets typically include an identical sequence L-2. Optionally, however, different label extenders (e.g., label extenders in different subsets) can include different sequences L-2.

In the embodiment depicted in FIG. 2, the label probe system includes the preamplifier, amplification multimer, and label probe. It will be evident that similar considerations apply to embodiments in which the label probe system includes only an amplification multimer and label probe or only a label probe.

The various hybridization steps can be performed simultaneously or sequentially, in any convenient order. For example, each target nucleic acid or capture probe can be hybridized simultaneously with its corresponding subset of m label extenders and its corresponding subset of amplifiers, and then the label probe system.

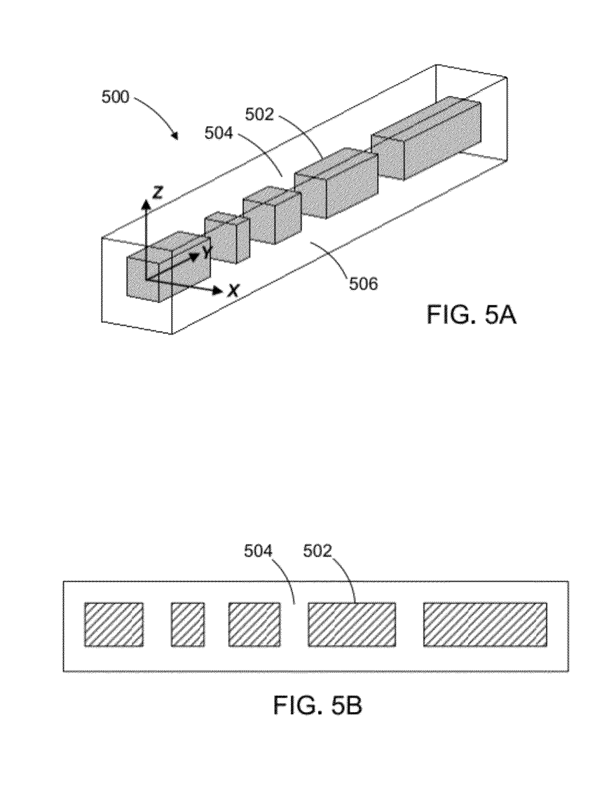

As previously mentioned, the solid support may be one or more particles, microparticles or nanoparticles. As an example, FIGS. 5A and 5B schematically illustrates an encoded microparticle which may be utilized as a substrate. Microparticle 500 is a cuboid structure elongated along the Y direction in the Cartesian coordinate as shown in the figure. The cross-sections perpendicular to the length of the microparticle have substantially the same topological shape—which is square in this example. The microparticle in this particular example has a set of segments (e.g. segment 502) and gaps (e.g. gap 504) intervening the segments. Specifically, segments with different lengths (the dimension along the length of the microparticle, e.g. along the Y direction) represent different coding elements; whereas gaps preferably have the same length for differentiating the segments during detection of the microparticles. The segments of the microparticle in this example are fully enclosed within the microparticle, for example within body 106. As an alternative feature, the segments can be arranged such that the geometric centers of the segments are aligned to the geometric central axis of the elongated microparticle. A particular sequence of segments and gaps represents a code. The codes are derived from a pre-determined coding scheme.

Segments of the microparticle can be any suitable form. For instance, each segment of the microparticle may have a substantially square cross-section (i.e. the cross-section in the X-Z plane of a Cartesian coordinate as shown in FIG. 5A) taken perpendicular to the length (i.e. along the Y direction in the Cartesian coordinate in FIG. 5A) of the microparticle. The segments may or may not be fabricated to have substantially square cross-section. Other shapes, such as rectangular, circular, and elliptical, jagged, curved or other shapes are also applicable. In particular, the code elements—i.e. segments and gaps, may also take any other suitable desired shape. For example, the segment (and/or the gaps) each may have a cross-section that is rectangular (e.g. with the aspect ratio of the rectangular being 2:1 or higher, such as 4:1 or higher, 10:1 or higher, 20:1 or higher, or even 100:1 or higher, but preferably less than 500:1). The code elements, i.e. the segments and gaps, may take any desired dimensions. As an example, each coding structure may have a characteristic dimension that is 5 nm (microns) or less, such as 3 microns or less, and more preferably 1 micron or less, such as 0.8 or 0.5 microns or less. In particular, when gaps are kept substantially the same dimension while the segments vary in dimension, each gap preferably has a characteristic dimension that is 1.5 microns or less, such as 0.8 or 0.5 microns or less. As one example, if forming the microparticles on a 12-inch silicon wafer with 0.13 line widths, the gap areas can be made to have 0.13 μm minimum widths, with the less transparent segments having widths of from 0.13 μm to much larger (depending upon the desired length of the particle and the encoding scheme and code space desired). Minimum gap widths, as well as minimum segment widths, of from 0.13 to 1.85 μm (e.g. from 0.25 to 0.85 μm) are possible depending upon the wafer fabrication used. Of course larger minimum gap and segment lengths (e.g. 1.85 to 5.0 μm, or more) are also possible. Other sized wafers (4 inch, 6 inch, 8 inch etc.) can of course be used, as well as wafers other than silicon (e.g. glass), as well as other substrates other than silicon (larger glass panels, for example).

The microparticle can have any suitable number of coding structures depending upon the shape or length of the particle, and the code space desired. Specifically, the total number of coding structures of a microparticle can be from 1 to 20, or more typically from 3 to 15, and more typically from 3 to 8. The desired code can be incorporated in and represented by the microparticle in many ways. As an example, the coding elements of the pre-determined coding scheme can be represented by the segment(s)—e.g. segments of different lengths represent different coding elements of the coding scheme. Different spatial arrangements of the segments with the different (or the same) lengths and intervened by gaps represent different codes. In this code-incorporation method, the intervening gaps preferably have substantially the same dimension, especially the length in the direction to which the segments are aligned. As another example, the codes are incorporated in the microparticle by arranging gaps that vary in lengths; while the segments have substantially the same dimension and are disposed between adjacent gaps. In another example, the both segments and gaps vary in their dimensions so as to represent a code. In fact, the code can also be represented in many other alternative ways using the segments, gaps, and the combination thereof. The particle code space may be further expanded by manufacturing a subset of the microparticles such that a tab protrudes from a face of the particle. Further, the code may also incorporate refractive or reflective coatings to expand the maximum number of allowable codes.

To enable detection of codes incorporated in microparticles, the segments and gaps in each microparticle can be composed of materials of different optical, electrical, magnetic, fluid dynamic, or other desired properties that are compatible with the desired detection methods. In one example the segments and gaps are directly spatially distinguishable under transmitted and/or reflected light in the visible spectrum. For example, when the code detection relies upon optical imaging, the distinguishable property (segments vs. gaps) can be a difference in transmissivity to the particular light used for imaging (which can be any desired electromagnetic radiation—e.g. visible and near-visible light, IR, and ultra-violet light. The segments can be made to be more light absorbing (or light reflecting) than the intervening spacing material (or vice versa). Regardless of which specific property is relied upon, the segments and gaps are preferred to exhibit sufficient difference in the specific property such that the difference is detectable using the corresponding code detection method. In particular, when the code is to be detected by means of optical imaging, the segments and gaps are composed of materials exhibiting different transmissivity (in an optical transmittance mode) or reflectivity (in optical reflectance mode) to the specific light used in imaging the microparticles. For example, the segments of the microparticle of the less transparent material can block and/or reflect 30% or more, preferably 50% or more, or e.g. 80% or more, of the visible light or near visible light incident thereon.

The microparticles may be made of organic and/or inorganic materials or a combination of organic and inorganic material. Specifically, the gaps (which are preferably more transmissive to visible or near-visible light) and segments (which are preferably less transmissive to visible or near-visible light as compared to gaps) each can be composed organic or inorganic materials, or a hybrid organic-inorganic material. The segments can be composed of a metal (e.g. aluminum), an early transition metal (e.g. tungsten, chromium, titanium, tantalum or molybdenum), or a metalloid (e.g. silicon or germanium), or combinations (or nitrides, oxides and/or carbides) thereof. In particular, the segments can be composed of a ceramic compound, such as a compound that comprises an oxide of a metalloid or early transition metal, a nitride of a metalloid or early transition metal, or a carbide of a metalloid or early transition metal. Early transition metals are those from columns 3b Sc, Y, Lu, Lr), 4b (Ti, Zr, Hf, Rf), 5b (V, Nb, Ta, Db), 6b (Cr, Mo, W, Sg) and 7b (Mn, Tc, Re, Bh) of the periodic table. However, preferred are early transition metals in columns 4b to 6b, in particular tungsten, titanium, zirconium, hafnium, niobium, tantalum, vanadium and chromium. Alternatively, the particles may be entirely comprised of different forms of silica, glass, or suitable known polymeric materials. The gaps which are in this example more transparent, can comprise any suitable material that is more transparent than the segments. The spacing material can be a siloxane, siloxene or silsesquioxane material, among others, if a hybrid material is selected. The spacing material, if inorganic, can be a glass material. Thin film deposited silicon dioxide is a suitable material, with or without boron or phosphorous doping/alloying agents. Other inorganic glass materials are also suitable such as silicon nitride, silicon oxynitride, germanium oxide, germanium oxynitride, germanium-silicon-oxynitride, or various transition metal oxides for example. A spin on glass (SOG) could also be used. If an organic material is used for the gap material, a plastic (e.g. polystyrene or latex for example) could be used. Both the segments and the gaps can be deposited by any suitable methods such as CVD (chemical vapor deposition), PVD (physical vapor deposition), spin-on, sol gel, etc. If a CVD deposition method is used, the CVD could be LPCVD (low pressure chemical vapor deposition), PECVD (plasma enhanced chemical vapor deposition), APCVD (atmospheric pressure chemical vapor deposition), SACVD (sub atmospheric chemical vapor deposition), etc. If a PVD method is used, sputtering or reactive sputtering are possible depending upon the desired final material. Spin on material (SOG or hybrid organic-inorganic siloxane materials

Other aspects of the microparticles are disclosed in the specification of U.S. patent application Ser. No. 11/521,057, especially at, for instance, sections entitled “Frabrication”, “Detection,” “Method for Producing Codes,” “Coding Scheme,” “Assays,” “A Bioassay Process Using the Microparticles,” and Figures, etc., all of which is incorporated herein by reference for all purposes.

Typically, the one or more cells of interest comprise two or more cells of interest, and the one or more subsets of m label extenders comprise two or more subsets of m label extenders.

In one class of embodiments in which the one or more different types of cells of interest comprise two or more different types of cells of interest and the one or more subsets of m label extenders comprise two or more subsets of m label extenders, a pooled population of particles is provided. The population comprises two or more subsets of particles, and a plurality of the particles in each subset is distinguishable from a plurality of the particles in every other subset. (Typically, substantially all of the particles in each subset are distinguishable from substantially all of the particles in every other subset.) The particles in each subset have associated therewith a different capture probe.

Two or more subsets of n capture extenders, wherein n is at least two, are also provided. Each subset of n capture extenders is capable of hybridizing to one of the optional target nucleic acids, and the capture extenders in each subset are capable of hybridizing to one of the capture probes, thereby associating each subset of n capture extenders with a selected subset of the particles. Each of the optional target nucleic acids is hybridized to its corresponding subset of n capture extenders and the subset of n capture extenders is hybridized to its corresponding capture probe, thereby capturing the nucleic acid on the subset of particles with which the capture extenders are associated.

Another general class of embodiments provides methods of capturing a label to a nucleic acid of interest. In the methods, m label extenders, wherein m is at least two, are provided. The m label extenders are capable of hybridizing to the nucleic acid of interest. A label probe system comprising the label is also provided. A component of the label probe system is capable of hybridizing simultaneously to at least two of the m label extenders. Each label extender comprises a polynucleotide sequence L-1 that is complementary to a polynucleotide sequence in the nucleic acid of interest and a polynucleotide sequence L-2 that is complementary to a polynucleotide sequence in the component of the label probe system, and the m label extenders each have L-1 5′ of L-2 or wherein the m label extenders each have L-1 3′ of L-2. The nucleic acid of interest is hybridized to the m label extenders, and the label probe system is hybridized to the m label extenders at a hybridization temperature, thereby capturing the label to the nucleic acid of interest. Preferably, the hybridization temperature is greater than a melting temperature Tₘ of a complex between each individual label extender and the component of the label probe system.

Thus, the present embodiments utilize microparticles and bDNA components to label, essentially barcoding, cells and detection of the same cells. The components include bDNA assay components and microparticles. To “barcode” the cells of interest with the microparticle containing the spatial “barcode,” the microparticles have attached to them either antigens and/or antibodies which are specific for the cell type of interest, as discussed above. The same microparticles will have attached to them components of the bDNA assay to allow for optional labeling and signal amplification if desired. Thus, cells may be barcoded and detected a number of different ways. Upon incubation of cells with the microparticles, the microparticles may be read directly to determine the barcode. Alternatively, the microparticles may have attached to them capture probes, to which the various architectures of the bDNA assay may be assembled (either with an intervening target nucleic acid or without) to amplify a detectable signal. The label probe system may utilize any of a number of known detection systems based on, for instance, chromogenic, fluorescent, radioactive, phosphate-based or any other detectable label system amenable to use in the bDNA assay system outlined above, and combinations and mixtures thereof.

The microparticles, as also briefly mentioned, may be uniformly coated with a mixture of antigens, antibodies and capture probes, or combinations thereof. For instance, the microparticles may be uniformly coated with just antigen, just antibody, antigen and antibody, antigen and capture probe, antibody and capture probe, antigen and antibody and capture probe, etc. The various combinations thereof will be apparent to one of skill in the art. Likewise it will be apparent that a uniform coating of all chosen components attached to the microparticle is not necessary and is optional. Instead, the placement of antigen, antibody and/or capture probe may be random, ordered, spatially arranged, geometrically arranged, equatorially arranged, set apart by surface space with no attachments, contiguous patches of different components, etc. A myriad number of various arrangements may be possible. However, a specific embodiment employs a random and/or uniform attachment of antibodies, antigens and/or capture probes across the entire surface of all microparticles, making assembly of such microparticles quick and efficient by attaching the various components in a single attachment step through reactions at the silica surface, for instance, across the entire surface of the particle.

Likewise, as pointed out above, the order of operation of the various steps including cell incubation, assembly of optional bDNA components, etc. may be any ordered desired and optimum for the specific assay. In some instances it may be easiest and most effective to have the entire bDNA architecture pre-assembled prior to incubation with the cells. In other instances it may be found to work best when there is no bDNA components used or when the bDNA components including the label probe system is added after incubation with cells to attach the microparticles to the cells.

The microparticles, once attached, may remain attached to the cells in a stable manner due to interactions between antibody and antigen, and/or antigen and cell receptor components. The cells or tissue in which the cells reside may then be further manipulated and/or processed in additional optional steps as desired while the microparticles remain attached. For instance, the tissue may be homogenized and individual cells released and sorted according to attached microparticle barcode. These cells may be further manipulated by imaging, sorting, counting, quantitating, genome sequencing, and the like. For instance, once barcoded, the cells may be sorted based on the barcode, the label probe system signal, etc. Homogeneous aliquots of cells of interest may then be obtained which may then be further manipulated in additional optional biological experiments designed to study the specific cell type obtained through the present methodologies.

For example, an antibody directed to a specific T-cell receptor mutant may be obtained. Such monoclonal and polyclonal antibodies specific to particular extracellular receptor mutations are known. These antibodies may be attached to microparticles and a heterogeneous population of T-cells may be differentially labeled depending on the status of the sequence to which the antibody has highest affinity and/or specificity. Thus having labeled only the T-cells carrying the mutation to which the antibody is specific, these cells may be process by, for instance, a fluorescence-activated cell sorter. The barcoded cells may be labeled using the label probe system of the bDNA assay. The T-cells of interest may then be sorted into a test tube and further analyzed. The specific components of the T-cells of interest to be analyzed may include, for instance, mitochondrial components, cell structure and cytoskeleton components, membrane components, nuclear extracts, protein or enzyme components, genomic components, and the like. Optionally, the barcoded and sorted homogeneous aliquot of T-cells of interest thus obtained may then be injected in vivo into test subjects to determine their efficacy, life cycle, biological activity, and the like. Tissues of the in vivo test subject may then be extracted and analyzed, the T-cell of interest being able to be tracked by the presence of the barcode and the ability to specifically label the T-cells of interest using the bDNA assay and components thereof as described above. The T-cell cell type used in this embodiment is merely used as an illustrative example only. Other antibodies specific for other cell types, such as, for example, B-cells, lipocytes, macrophages, red blood cells, islet cells, chondrocytes, neural cells, stem cells, skin cells, muscle cells, fibroblasts, and the like may be equally utilized or substituted for the exemplary T-cells mentioned herein.

In another exemplary embodiment, a library of antibodies may be obtained, each specific for a sub-family of extracellular receptor sequence. A myriad such antibodies have been reported. Each antibody can be assigned a specific barcode or set of barcodes encoded by microparticles. Each such set of microparticles may have attached thereto specific capture probes which bind to different label probe systems yielding differentially detectable signals. In this manner, the present embodiments may be used to highlight in either circulating cells or whole tissues each specific family of sub-sequence desired to be detected. Again, such labeled samples may then be further manipulated by sorting, counting, and analyzing as mentioned above.

B. Immunocapture and Sorting of Cells Using the bDNA Assay

As previously mentioned, the QUANTIGENE® technology allows unparalleled signal amplification capabilities that provide an extremely sensitive assay. For instance, it is commonly claimed that the limit of detection in situ for mRNA species using methods other than the QUANTIGENE® technology is about 20 copies of message per cell. However, in practice the limit of detection, due to the variability in the assay, is generally found to be around 50-60 copies of message per cell. This limit of detection limits the field of research since 80% of mRNAs are present at fewer than 5 copies per cell and 95% of mRNAs are present in cells at fewer than 50 copies per cell. In contrast, the QUANTIGENE® technology, such as QUANTIGENE® 2.0 and ViewRNA, is very simple, efficient and is capable of applying up to 400 labels per 50 base pairs of target. This breakthrough technology allows efficient and simple detection on the level of even a single mRNA copy per cell. Coupling this technology to detection of both mRNA and protein species will propel this field of research into heretofor inaccessible areas of study.

An exemplary method involves the use of multiple technologies to achieve an unparalleled result in the research and diagnostic fields. In this embodiment of the present methods, any species of cell may be detected using techniques generally described in the Panomics website for QUANTIGENE® ViewRNA protocols, as mentioned above. The manual for this protocol, “QUANTIGENE® ViewRNA User Manual,” incorporated by reference in its entirety for all purposes, may also be downloaded from the Panomics website (see, panomics.com/downloads/UM15646—QGViewRNA_RevA_{—}080526.pdf, contents of which are incorporated herein by reference in its entirety for all purposes). Branched DNA technology is used, comprising pre-amplifiers, amplifiers and label probes, to amplify the signal associated with the captured target nucleic acids. To make the assay more robust, nucleic acid analogs are utilized in the capture extender probes, such as LNA, etc. This provides increase specificity for the target.

As a second layer to this, antibodies directed to a target cells of interest may be used, which have conjugated thereto a sequence of DNA similar to a pre-amplifier sequence which comprises A-1 sequences which are complementary to the A-2 sequences of matching amplifier probes (see, FIG. 8A). This then allows specific binding of, and tagging of, target cells of interest.

Additionally, nucleic acid analogs such as constrained-ethyl (cEt) analogs may be used. (See, FIGS. 4A and 4B, and for additional variations of this analog which may also be suitable in the present embodiments, Seth et al., “Short Antisense Oligonucleotides with Novel 2′-4′ Conformationaly Restricted Nucleoside Analogues Show Improved Potency Without Increased Cytotoxicity in Animals,” J. Med. Chem., 52(1):10-13, 2009, incorporated herein by reference in its entirety for all purposes). The pre-amplifier probe may be entirely comprised of such cEt analogs, or may be only partially comprised of cEt analogs. Specifically, the pre-amplifier conjugated to the antibody may only have cEt analogs at sequence A-1. Alternatively, or in addition, the label extender probe used to capture the RNA species may be entirely comprised of cEt analogs at the L-1 sequence. Use of the cEt analogs in the assay is especially beneficial because it is known that cEt analogs, when present in probes, act to increase the melting temperature of the resulting hybridized probe:target pair, which provides increased stability of the hybridized pair.

The length of label extender probes may vary in length anywhere from 10 to 60 nucleic acids or more, i.e. 11, 13, 15, 17, 19, 21, 25, 30, 35, 40, 45 or 50 nucleic acids in length. The sequence L-1 will also vary depending on the identity of the target and the number of potentially cross-reacting probes within the hybridization mixture. For instance, L-1 may be anywhere from 7 to 50 nucleic acids in length, or 10 to 40, or 12 to 30 or 15 to 20 nucleotides in length. The sequence L-1 may be entirely comprised of nucleic acid analogs or only partly comprised of nucleic acid analogs. For instance, it may be that every other nucleic acid is an analog in L-1, providing a 50% substitution of analog for native or wild type base. Alternatively, the L-1 sequence may be 100% comprised of nucleic acid analog. Further the L-1 sequence may be 10%, 20%, 30%, 40%, 50%, 60%, 70%, 80% or 90% comprised of nucleic acid analog. The underlying principle to the use of nucleotide analogs, such as cEt, is to increase the melting temperature or temperature at which the L-1 sequence remains hybridized to the target sequence. Typically, the LE and CE may be designed such that the target melting temperature for the assay is in the range of 50° C. to 56° C., or 49° C. to 57° C., or 48° C. to 48° C., etc. However, this may vary depending on buffer conditions and assay. For instance, when performing an in situ assay, it may be useful to add a neutralizing or denaturing agent such as formamide, and thereafter adjust the target melting temperature downwards to a range of 40° C. to 50° C. or lower. Thus the amount of melting temperature-increasing nucleotide analog present in L-1 can be doped up or down to the desired and empirically-determined most suitable amount to achieve the desired melting temperature, which will in turn provide the best performance with respect to affinity and specificity. Design of the L-1 sequence, as in any probe sequence binding to the target, and determination of the amount of nucleotide analog to use in a specific embodiment of the presently disclosed assays, will depend on many factors including target sequence, buffer conditions and melting temperature needed to achieve the desired specificity and affinity in the assay.

The length of the sequence covalently attached to the antibody may be of any suitable length. In general, the length may be sufficient for any suitable number of label extender probe pairs to bind to it. For instance, as mentioned above, stable capture of the component of the label probe system by the at least two label extenders, e.g., while minimizing capture of extraneous nucleic acids, can be achieved, for example, by balancing the number of label extenders that bind to the component, the amount of overlap between the label extenders and the component (the length of L-2 and M-1), and/or the stringency of the conditions under which the label extenders and the component are hybridized. For instance, when detecting a large message RNA of several hundred base pairs or less, any number of label extenders may be used, such as, for instance, 1-30 pairs of label extender probes, or 2-28 pairs of label extender probes, or 3-25 pairs of label extender probes, or 4-20 pairs of label extender probes, or a number of label extender probe pairs which is suitable to specifically attach the label probe system to the target with the desired affinity. The sequence covalently attached to the antibody may be comprised of RNA, DNA, or any analogues thereof as discussed above. The entirety of the sequence covalently attached to the antibody may be comprised of analog, or only certain percentages of the sequence may be comprised of analog. In general the sequence conjugated to the antibody may be anywhere from 100-200 base pairs in length.

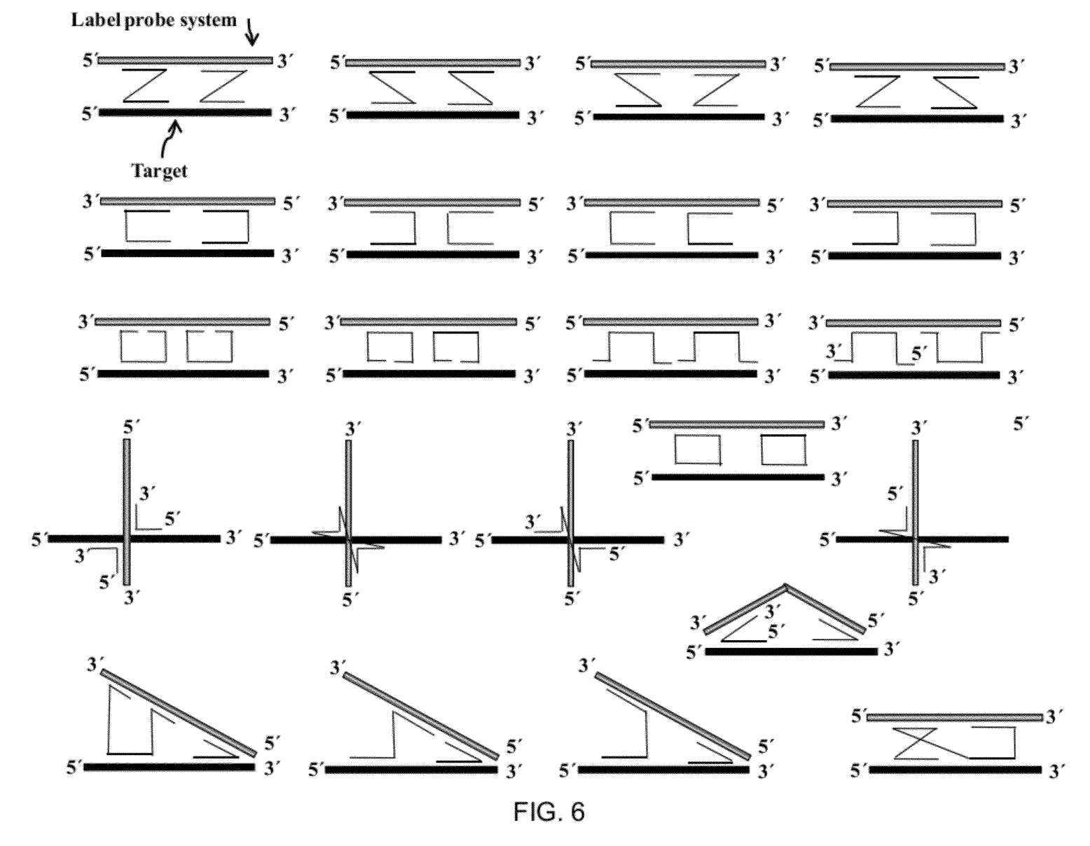

It is further noted that the label extenders, used to bind to the captured target nucleic acid and the pre-amplifiers, may be in any of many different conformations. That is, the label extenders may be designed in the double-z (ZZ) configuration, the cruciform configuration, or any other related conformation as depicted, for instance, in FIG. 6. Each of these interchangeable conformations may be designed and utilized in these assays to achieve similar results. The structural variations of label extender probe design depicted in FIGS. 7A and 7B are only non-limiting examples and the Figures do not depict all possible geometries or strategies. One of skill will recognize that other useful and suitable label extender probe designs may be derived from these exemplary structures. More specifically it has been determined that especially the ZZ and the cruciform conformations work well in these assays. Furthermore, it is noted that various geometric alignments may be utilized in designing the cruciform and ZZ conformations. FIGS. 7A and 7B are not intended to depict every possible design of the label extenders. Rather, these Figures merely depict specific embodiments of label extender design. One of skill in the art would be able to design other variations based on these themes which may also be suitable for the herein described methodological embodiments.

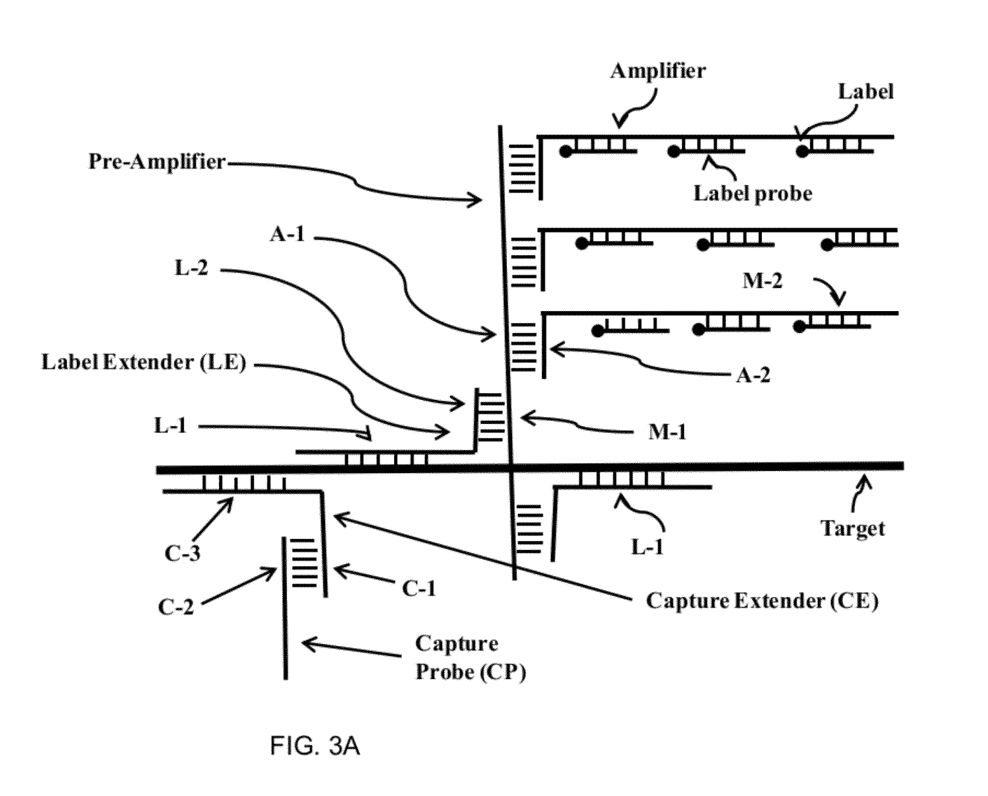

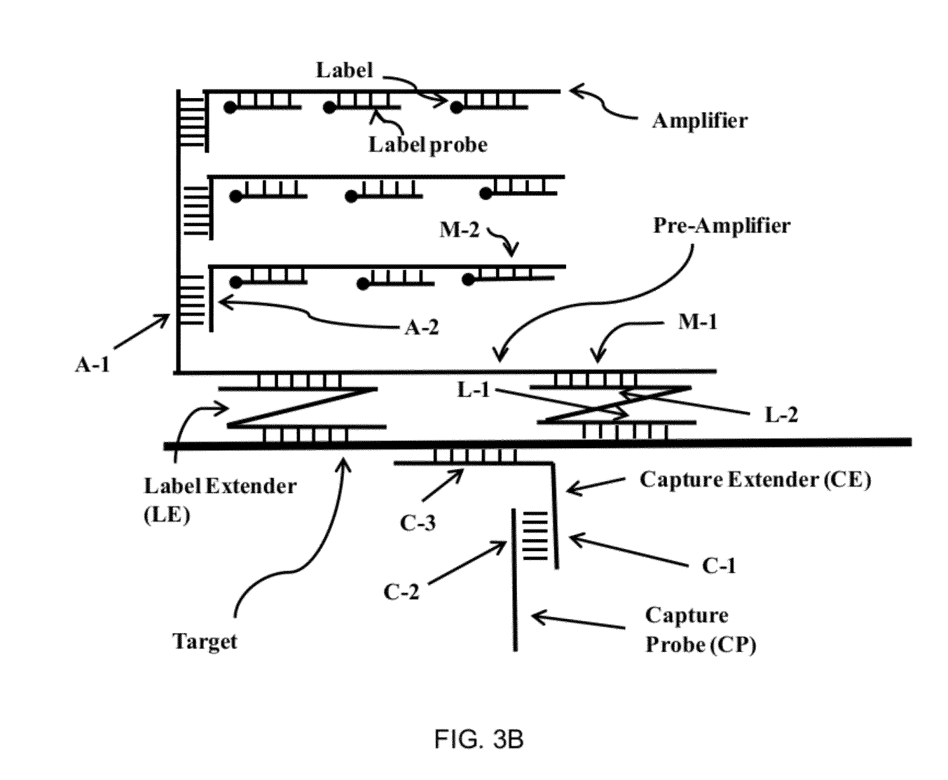

This embodiment may be used to detect as many target cells of interest as desired, corresponding to the number of different labels are available. Labels have been mentioned elsewhere in the present application and may be used in combination to label each species with a different observable signal, such that multiple proteins and nucleic acid species may be simultaneously detected. The label extenders are therefore designed to bind to their respective specific L-1 complementary regions (L-2) on the target nucleic acid, while amplifier probes specific for the pre-amplifier binding to that label extender pair will only bind labels of one type, as illustrated in FIGS. 3A and 3B. Meanwhile, the pre-amplifier probe conjugated to the antibody, or antibodies, will comprise specific A-1 sequences, different from the A-1 sequences of the pre-amplifier binding the label extender probes, which bind only amplifiers which in turn have sequences which only the second (or third, or fourth, etc.) label probes will bind. Thus, a specific type of label signal may be associated with a specific cell type of interest which has expressed on its surface the target protein of interest, and a second distinguishable type of label may be associated with a different target protein of interest. As many probes may be designed as needed, such that multiple proteins may be simultaneously associated with specific label probe systems in a single assay, enabling multiplexed detection. That is, this approach enables multiplex detection of multiple antigens/proteins in a single assay. Further, the present embodiment may be amenable to in situ procedures, in cellulo procedures using purified cells from tissue culture, or even FFPE samples under proper conditions.

Further, cross-linking of the label extender probes or antibodies to the targets will improve reproducibility and sensitivity. Such cross-linking methods may be utilized in the present embodiment, as well as in other embodiments, for instance in labeling of cells using microparticles, as discussed above. Various known chemical cross-linking agents may be adapted to the protocol to aid in more permanently fixing the label probe system of QUANTIGENE® to the tissues or cells, such as, for instance, carbodiimides such as 1-ethyl-3-[3-dimethylaminopropyl]carbodiimide hydrochloride (EDC) (see, for instance, Nat. Protoc., 3(6):1077-1084, 2008 and Nuc. Acids Res., 38(7):e98, 2010 both of which are incorporated herein by reference for all purposes) and similar amine-to-carboxyl cross linkers known in the art (see, for instance, Pierce Cross-Linking Reagents Technical Handbook, from Pierce Biotechnology, Inc., 2005, available for download from the internet at the Pierce website, at (www.) piercenet.com/Files/1601673_CrosslinkHB_Intl.pdf, incorporated herein by reference for all purposes), or other suitable cross-linkers as may be determined empirically, such as carboxyl-carboxyl, carboxyl-amine and amine-amine cross linking reagents, for instance such as those listed in the Pierce Biotechnology, Inc. catalogs. Other methods for cross linking known in the art include, but are not limited to, the use of Br-dU and/or I-dU modified nucleic acids where the 5-methyl group on the U base is substituted for the atom Br or I and crosslinking is triggered by irradiation at 308 nm. (See, Willis et al., Science, 262:1255, 1993). Other useful crosslinking agents may include psoralens which intercalate between bases and upon irradiation at 350 nm covalent crosslinking occurs between thymidine bases, which is reversible when irradiated again at 254 nm. (See, Pieles et al., Nuc. Acid Res., 17:285, 1989). These and other crosslinkers of the same family and of other well known families may be useful in achieving the same or similar results, i.e. stabilizing the interaction between the label probe system components and/or antibodies and the target nucleic acids and proteins by forming a covalent bond between the two species of molecules. One of skill in the art is generally familiar with various protocols for achieving such cross-linking.

In another embodiment, the present components may be manipulated to achieve detection of miniscule amounts of antigen (e.g. extracellular protein expressed by the cell of interest) in any sample. As discussed above, the limits of detection may be amplified 400-fold or more using the presently disclosed components. By covalently conjugating a pre-amplifier probe to an antibody, any antigen may be detectable using the present systems. In the present embodiment, it is possible to assign each available antibody to a different pre-amplifier comprising different A-1 sequences, each binding a different amplifier and a different label probe. Any number of different antibody species may be utilized in the present embodiment. For instance, as mentioned above, various forms of antibodies are known in the art, such as diabodies, triabodies, minibodies, antibody fragments and even molecules that mimic antibodies. In short, any molecule capable of being conjugated to a pre-amplifier of the present label probe system may be used in the present embodiment to detect the antigen to which it binds. For instance, antigens, agonists and antagonists, i.e. those proteins which bind to extracellular receptors, may be conjugated to pre-amplifiers in the same manner, as well as sugar binding proteins, e.g. lectins, leptins and the like, phosphate-binding proteins, and the like, all may be utilized in like manner instead of antibodies.

Various methods of conjugating DNA sequences to antibodies are known in the art. Such methods are known and are capable of creating a covalent bond between a component of the DNA sequence and a component of the antibody. However, alternatives to conjugation are also known, such as the use of strong affinity interactions such as avidin-biotin interactions. Avidin and biotin may be covalently associated with either antibody or pre-amplifier to achieve association of the amplifier probes and the label probe system to the antibody or similar molecule having a specific affinity for an antigen or agonist/antagonist or the like, and therefore to each different antigen or agonist/antagonist or binding partner and the like.

### C. Compositions

Compositions related to the methods are another feature of the invention. Thus, one general class of embodiments provides a composition for detecting two or more nucleic acids of interest. In one aspect, the composition includes a pooled population of particles. The population comprises two or more subsets of particles, with a plurality of the particles in each subset being distinguishable from a plurality of the particles in every other subset. The particles in each subset may have associated therewith a different capture probe. In alternative embodiments, the microparticles may have optionally attached to them antigens, antibodies and/or capture probes, and mixtures and combinations thereof. That is, the microparticles may be present in a composition having attached thereto ahead of time, as if part of a kit, the specific antibodies, antigens and/or capture probes desired for the assay and specific for the types of cells of interest.

The composition also optionally may include two or more subsets of n capture extenders, wherein n is at least two, two or more subsets of m label extenders, wherein m is at least two, and a label probe system comprising a label, wherein a component of the label probe system is capable of hybridizing simultaneously to at least two of the m label extenders in a subset. Each subset of n capture extenders is capable of hybridizing to one of the target nucleic acids, and the capture extenders in each subset are capable of hybridizing to one of the capture probes and thereby associating each subset of n capture extenders with a selected subset of the particles. Similarly, each subset of m label extenders is capable of hybridizing to one of the target nucleic acids, if such is used in the assay.

The composition optionally includes a sample comprising or suspected of comprising at least one of the cells of interest, e.g., two or more, three or more, etc. cell types of interest. Optionally, the composition comprises one or more of the cell types of interest. In one class of embodiments, each target nucleic acid is hybridized to its corresponding subset of n capture extenders, and the corresponding subset of n capture extenders is hybridized to its corresponding capture probe. Each cell type of interest is thus associated with an identifiable subset of the particles. In this class of embodiments, each target nucleic acid present in the composition is also hybridized to its corresponding subset of m label extenders. The component of the label probe system (e.g., the amplification multimer or preamplifier) is hybridized to the m label extenders. The composition is maintained at a hybridization temperature that is greater than a melting temperature Tₘ of a complex between each individual label extender and the component of the label probe system (e.g., the amplification multimer or preamplifier). The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the Tₘ.

Compositions may also optionally comprise antibodies specific for various antigens of interest and/or agonists or antagonists which bind to extracellular receptors of interest, for example. Compositions may also comprise antibodies pre-conjugated to docking sequences of various lengths capable of hybridizing to L-1 regions of included matching label extender probe pairs for signal amplification.

Another general class of embodiments provides a composition for detecting one or more cell type of interest. The composition includes microparticles comprising one or more capture probes, one or more subsets of n capture extenders, wherein n is at least two, one or more subsets of m label extenders, wherein m is at least two, and a label probe system comprising a label. Each subset of n capture extenders is capable of hybridizing to one of the nucleic acids of interest, and the capture extenders in each subset are capable of hybridizing to one of the capture probes and thereby associating each subset of n capture extenders with the microparticles. Each subset of m label extenders is capable of hybridizing to one of the target nucleic acids, if used. A component of the label probe system (e.g., a preamplifier or amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2.

In one class of embodiments, the one or more cell types of interest comprise two or more cell types of interest, the one or more subsets of n capture extenders comprise two or more subsets of n capture extenders, the one or more subsets of m label extenders comprise two or more subsets of m label extenders, and a pooled population of particles. The population comprises two or more subsets of particles. A plurality of the particles in each subset are distinguishable from a plurality of the particles in every other subset, and the particles in each subset have associated therewith a different capture probe. The capture extenders in each subset are capable of hybridizing to one of the capture probes and thereby associating each subset of n capture extenders with a selected subset of the particles.

For example, the label probe system can include an amplification multimer or preamplifier, which amplification multimer or preamplifier is capable of hybridizing to the at least two label extenders. The composition optionally includes one or more of the target nucleic acids, wherein each target nucleic acid is hybridized to its corresponding subset of m label extenders and to its corresponding subset of n capture extenders, which in turn is hybridized to its corresponding capture probe. The amplification multimer or preamplifier is hybridized to the m label extenders. The composition is maintained at a hybridization temperature that is greater than a melting temperature Tₘ of a complex between each individual label extender and the amplification multimer or preamplifier (e.g., about 5° C. or more, about 7° C. or more, about 10° C. or more, about 12° C. or more, about 15° C. or more, about 17° C. or more, or about 20° C. or more greater than the Tₘ).

Compositions are also understood to comprise label extenders and capture extenders having one or more nucleic acid analogs. That is, the sequences of L-1 and C-3, may contain anywhere from 1% to 100% nucleic acid analogs, such as, for instance, cEt, LNA, PNA and the like, and mixtures thereof. With regard to cEt, it is understood that other nucleic acid analogs of similar structure and having the same or similar properties, i.e. the ability to increase the melting temperature of a hybridization event between the capture extender and/or label extender sequence and the target sequence (see, for instance, FIGS. 4A and 4B). Thus, minor alterations to the structure of the cEt, including, but not limited to, addition of other alkyl groups, alkylene groups, thiols, amines, carboxyls, etc. which have similar chemical properties suitable to the assays and methods provided above, are also included in these compositions. Compositions are further intended to include those compositions designed specifically for detection of target nucleic acids in situ, which would not require the use of, and therefore not include in the composition, capture probes, capture extenders and/or particles.

### D. Labels

A wide variety of labels are well known in the art and can be adapted to the practice of the present invention. For example, luminescent labels and light-scattering labels (e.g., colloidal gold particles) have been described. (See, e.g., Csaki et al. (2002) “Gold nanoparticles as novel label for DNA diagnostics,” Expert Rev. Mol. Diagn., 2:187-93).

As another example, a number of fluorescent labels are well known in the art, including but not limited to, hydrophobic fluorophores (e.g., phycoerythrin, rhodamine, Alexa Fluor 488 and fluorescein), green fluorescent protein (GFP) and variants thereof (e.g., cyan fluorescent protein and yellow fluorescent protein), and quantum dots. (See, e.g., The Handbook: A Guide to Fluorescent Probes and Labeling Technologies, Tenth Edition or Web Edition (2006) from Invitrogen (available on the internet at probes.invitrogen.com/handbook), for descriptions of fluorophores emitting at various different wavelengths (including tandem conjugates of fluorophores that can facilitate simultaneous excitation and detection of multiple labeled species). For use of quantum dots as labels for biomolecules, see e.g., Dubertret et al. (2002) Science, 298:1759; Nature Biotechnology (2003) 21:41-46; and Nature Biotechnology (2003) 21:47-51. Other various labels are known in the art, such as Alexa Fluor Dyes (Life Technologies, Inc., California, USA, available in a wide variety of wavelengths, see for instance, Panchuk, et al., J. Hist. Cyto., 47:1179-1188, 1999), biotin-based dyes, digoxigenin, AttoPhos (JBL Scientific, Inc., California, USA, available in a variety of wavelengths, see for instance, Cano et al., Biotechniques, 12(2):264-269, 1992), etc.

Labels can be introduced to molecules, e.g. polynucleotides, during synthesis or by postsynthetic reactions by techniques established in the art; for example, kits for fluorescently labeling polynucleotides with various fluorophores are available from Molecular Probes, Inc. ((www.) molecularprobes.com), and fluorophore-containing phosphoramidites for use in nucleic acid synthesis are commercially available. Similarly, signals from the labels (e.g., absorption by and/or fluorescent emission from a fluorescent label) can be detected by essentially any method known in the art. For example, multicolor detection, detection of FRET, fluorescence polarization, and the like, are well known in the art. As previously mentioned, labels also include lanthanide-based labels, such as those offered by DVS Sciences, Inc. (Sunnyvale, Calif.).

### E. General Microbiology Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., Current Protocols, a joint venture between Greene Publishing Associates, Inc. and John Wiley & Sons, Inc., (supplemented through 2006). Other useful references, e.g. for cell isolation and culture (e.g., for subsequent nucleic acid or protein isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla.

### F. Polynucleotide Synthesis

Methods of making nucleic acids (e.g., by in vitro amplification, purification from cells, or chemical synthesis), methods for manipulating nucleic acids (e.g., by restriction enzyme digestion, ligation, etc.) and various vectors, cell lines and the like useful in manipulating and making nucleic acids are described in the above references. In addition, methods of making branched polynucleotides (e.g., amplification multimers) are described in U.S. Pat. No. 5,635,352, U.S. Pat. No. 5,124,246, U.S. Pat. No. 5,710,264, and U.S. Pat. No. 5,849,481, as well as in other references mentioned above.

In addition, essentially any polynucleotide (including, e.g., labeled or biotinylated polynucleotides) can be custom or standard ordered from any of a variety of commercial sources, such as The Midland Certified Reagent Company ((www.) mcrc.com), The Great American Gene Company ((www.) genco.com), ExpressGen Inc. ((www.) expressgen.com), Qiagen (oligos.qiagen.com) and many others.

A label, biotin, or other moiety can optionally be introduced to a polynucleotide, either during or after synthesis. For example, a biotin phosphoramidite can be incorporated during chemical synthesis of a polynucleotide. Alternatively, any nucleic acid can be biotinylated using techniques known in the art; suitable reagents are commercially available, e.g., from Pierce Biotechnology ((www.) piercenet.com). Similarly, any nucleic acid can be fluorescently labeled, for example, by using commercially available kits such as those from Molecular Probes, Inc. ((www.) molecularprobes.com) or Pierce Biotechnology ((www.) piercenet.com) or by incorporating a fluorescently labeled phosphoramidite during chemical synthesis of a polynucleotide.

G. bDNA Labeling of Cells Using Lanthanide Labels

The label probes of the present invention may be comprised of any label desired, as mentioned above. Lanthanides offer several advantages over other labels in that they are stable isotopes, there are a large number of them available, up to 100 or more distinct labels, they are relatively stable, and they are highly detectable and easily resolved between detection channels when detected using mass spectrometry. Lanthanide labels also offer a wide dynamic range of detection. Lanthanides exhibit high sensitivity, are insensitive to light and time, and are therefore very flexible and robust and can be utilized in numerous different settings.

Lanthanides are a series of fifteen metallic chemical elements with atomic numbers 57-71. They are also referred to as rare earth elements. Lanthanides may be detected using CyTOF technology. CyTOF is inductively coupled plasma time-of-flight mass spectrometry (ICP-MS). CyTOF instruments are capable of analyzing up to 1000 cells per second for as many parameters as there are available stable isotope tags.

The lanthanide labels can be chelated by metal chelators attached to antibodies, such as those sold by DVS Sciences, Inc. Otherwise, the metal chelators may be attached to label probes of the bDNA system to allow attachment of lanthanides to the label tree created by the hybridization of the various structures of the bDNA system described above.

In cellular-based assays, where cells are being labeled, antibodies coupled to the lanthanides are bound to the cells. The antibodies specifically recognize surface markers on the cells of interest. The labeled cells are then subjected to CyTOF where the presence of the metal tag is indicative of the presence of the antibody and therefore the surface marker. Each cell is individually subjected to an inductively coupled plasma, atomizing and ionizing the cells. Atomic ions are then detected by the mass spectrometer. Only cells possessing a lanthanide attached to it may be detected. In this manner, the cells may actually be quantitated since the signal retrieved is linear with cell number.

Thus, at least two uses of lanthanides are contemplated. First, lanthanides may be used to label the label probes in the bDNA system. As in the QUANTIGENE® View system (Affymetrix, Inc., Santa Clara, Calif.), the bDNA probes may be added to tissue or cell culture. In tissue culture, if detecting RNA, the RNA could be dual labeled. The RNA would first be labeled normally with fluorescent labels or some other label, perhaps a colorimetric label, in order to localize the target in the tissue and image the tissue. Once imaged, the fluorescent labels can be washed off and the lanthanide labels applied. Laser dissection may be employed to specifically remove the cells of interest which are then fed into the cyTOF machine and analyzed.

In a second embodiment of the lanthanide label, again the lanthanide labels would be attached to label probes of the bDNA QuantiGene® system and used to label intact cells. That is, the cells are made permeable and the various probes and architecture needed to conduct the bDNA assays are inserted into the cell. Again, once the cells have been labeled, they would be processed as above by submission to a CyTOF analysis.

The CyTOF instrument is available from DVS Sciences, Inc. (Sunnyvale, Calif.). (See, Cheung et al., “Screening: CyTOF—the next generation of cell detection,” Nature Reviews Rheumatology, 7:502-503, 2011, and Bendall et al., “Single-Cell Mass Cytometry of Differential Immune and Drug Responses Across a Human Hematopoietic Continuum,” Science, 332(6030):687-696, 2011, incorporated herein by reference).

H. bDNA Assays on Microarrays

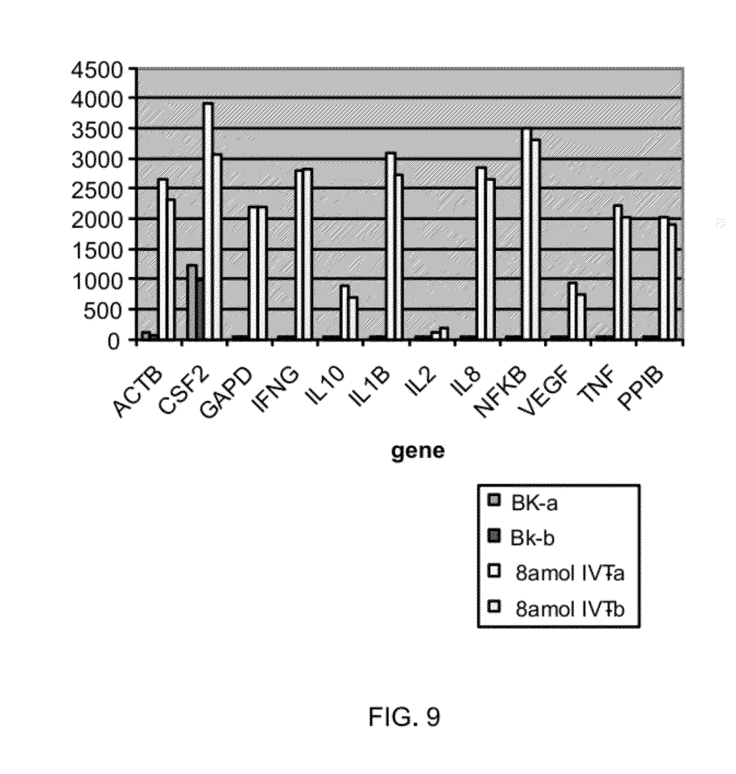

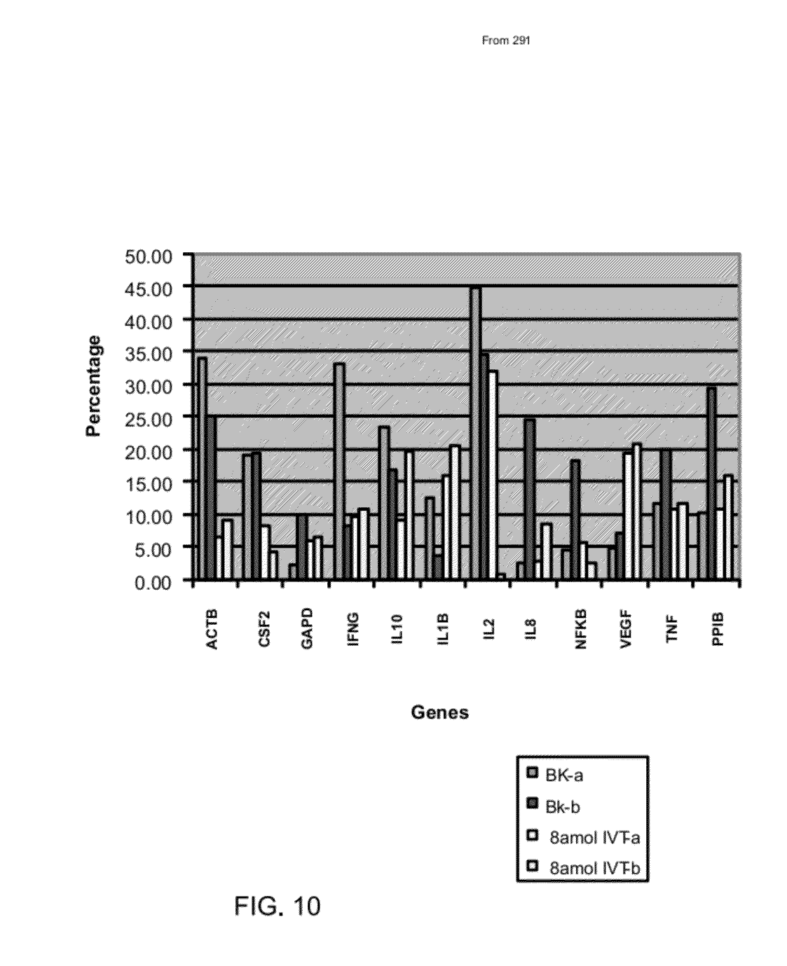

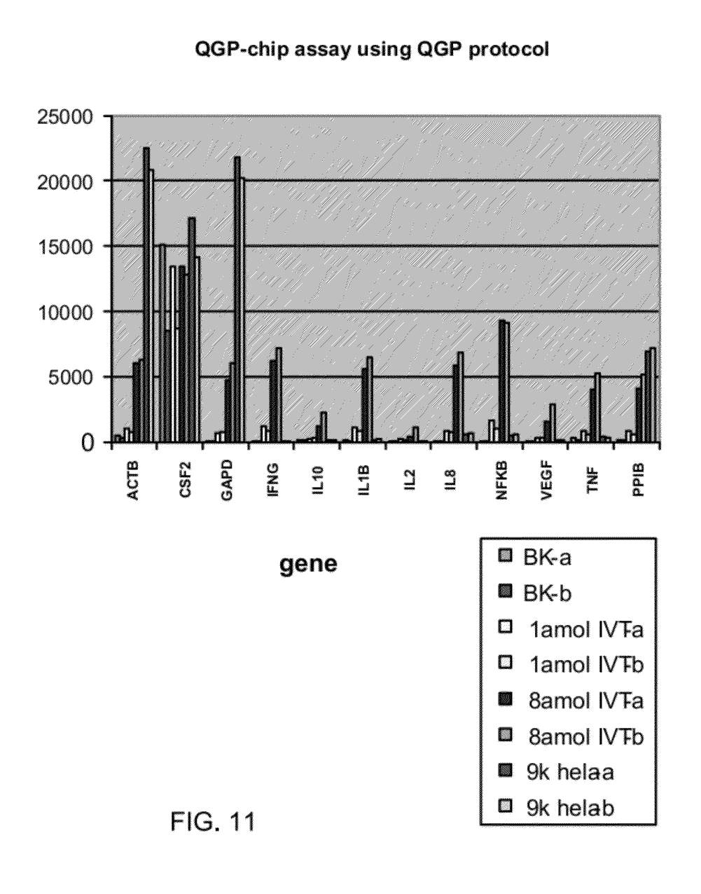

QuantiGene® plex assay (Affymetrix, Inc., Santa Clara, Calif.) is performed on the Luminex system. This system allows for up to about 50 plex. By combining QuantiGene® technology and the nucleotide microarray technology, up to 200 QuantiGene® plex can be performed. Affymetrix offers fully automated systems (such as the GeneTitan®, etc.) and high throughput assay systems that are based on nucleotide microarray assays. (See, FIGS. 9-11).

The bDNA QuantiGene® assay has been performed at the level of 12-plex, i.e. twelve different targets were detected, using the Affymetrix 3k tag chip. The data indicate that this assay has equivalent sensitivity, linearity, robustness as compared to QuantiGene® plex run on the Luminex system. Targets included the genes for ACTB, CSF2, GAPD, IFN-gamma, IL-10, IL-1B, 1L-2, IL-8, NFk-B, VEGF, TNF, and PPIB. Biotin-SAPE was used as the label. Assays were performed using cell lysates as the sample. The GCS3000 Affymetrix instrument was used to scan and analyze the microarray data according to published protocols. Essentially, the probes on the microarray behaved in the bDNA assay as the capture extender probes. Thus, the microarray probes were designed to hybridize to target. The end of the microarray probe not bound to the silicon chip is the end that has a stretch of nucleotides that are complimentary to non-overlapping segments of the target sequence. In this way, the target nucleic acid sequence is captured by the microarray, across several different microarray-bound probes acting as CEs. The label extenders and amplifiers are then added with label probes to provide signal amplification. Initial tests indicate that this method works well, is robust and has good linearity and sensitivity.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of labeling a cell, which comprises:
providing a sample comprising or suspected of comprising a cell;
incubating a microparticle with the sample, wherein the microparticle comprises a spatial code, a protein having affinity for an extracellular protein and a capture probe such that the protein binds the extracellular protein thereby associating the microparticle with the cell; and
incubating one or more label extender probes and a label probe system with the sample such that the one or more label extender probes and label probe system hybridize with the capture probe, wherein the label extender probes comprise a sequence L-1 which is complementary to a sequence in the capture probe and a sequence L-2 complementary to a sequence found in a component of the label probe system, thereby labeling the cell.

2. The method according to claim 1, wherein the sample comprises or is suspected of comprising at least two different cells and the label probe system comprises at least two different labels, each specific for each cell.

3. The method according to claim 1, wherein the label attached to the cells is detected by flow cytometry.

4. The method according to claim 1, wherein the cells are non-adherent and circulating cells.

5. The method according to claim 1, wherein quantity of the label is detected, thereby quantitating the number of cells labeled.

6. The method according to claim 1, wherein the protein having affinity for an extracellular protein is an antibody.

7. The method according to claim 1, wherein the protein having affinity for an extracellular protein is an antigen.

8. The method according to claim 1, wherein the protein having affinity for an extracellular protein is selected from one or more of the group consisting of: agonist, antagonist, phosphate-binding protein, saccharide-binding protein, and leptin-binding protein.

9. The method according to claim 1, wherein the spatial code of the microparticle is discernable by visual inspection.

10. The method according to claim 1, wherein the cell is selected from one or more of the group consisting of: stem cell, fibroblast, red blood cell, T cell, B cell, macrophage, lymphocyte, adipose cell, chondrocyte, and white blood cell and mixtures and combinations thereof.

11. A method of labeling a cell, which comprises:
providing a sample comprising or suspected of comprising a cell;
incubating a protein having affinity for an extracellular protein with the sample such that the protein having affinity for an extracellular protein binds to the cell, wherein the protein having affinity for an extracellular protein comprises at least one amplifier or pre-amplifier probe sequence; and
incubating at least one label probe system with the sample, thereby labeling the cell.

12. The method according to claim 11, wherein the protein having affinity for an extracellular protein is an antibody or an antigen.

13. The method according to claim 12, wherein the antibody or antigen is specific for an extracellular protein of the cell.

14. A method of labeling a cell, which comprises:
providing a sample comprising or suspected of comprising a cell;
incubating a microparticle comprising a spatial code with the sample, wherein the microparticle comprises a protein having affinity for an extracellular protein and one or more capture probes, such that the microparticle binds to the cell;
incubating the sample with one or more capture extenders, one or more target nucleic acids, one or more label extenders and one or more label probe systems such that the cell bound to the microparticle is labeled thereby labeling the cell.

15. The method according to claim 14, wherein the sample comprises or is suspected of comprising at least two different cells and the label probe system comprises at least two different labels, each specific for each cell.

16. The method according to claim 14, wherein the label attached to the cells is detected by flow cytometry.

17. The method according to claim 14, wherein the cells are non-adherent and circulating cells.

18. The method according to claim 14, wherein quantity of the label is detected, thereby quantitating the number of cells labeled.

19. The method according to claim 14, wherein the protein having affinity for an extracellular protein is an antibody.

20. The method according to claim 14, wherein the protein having affinity for an extracellular protein is an antigen.

21. The method according to claim 14, wherein the protein having affinity for an extracellular protein is selected from one or more of the group consisting of: agonist, antagonist, phosphate-binding protein, saccharide-binding protein, and leptin-binding protein.

22. The method according to claim 14, wherein the spatial code of the microparticle is discernable by visual inspection.

23. The method according to claim 14, wherein the cell is selected from one or more of the group consisting of: stem cell, fibroblast, red blood cell, T cell, B cell, macrophage, lymphocyte, adipose cell, chondrocyte, and white blood cell.
